(19) United States
(12) Patent Application Publication
(10) Pub. No.: US 2023/0310155 A1
(43) Pub. Date: Oct. 5, 2023

(54) MULTI-ANCHOR DELIVERY SYSTEMS

(71) Applicant: EDWARDS LIFESCIENCES CORPORATION, Irvine, CA (US)

(72) Inventors: Stephen Cournane, Severn, MD (US); Wesley Paul Nicholson, Sunderland, MD (US); Harvey H. Chen, Irvine, CA (US); Maria L. Saravia, Irvine, CA (US); Derrick Johnson, Orange, CA (US); Felino V. Cortez, JR., Bowie, MD (US); Stephen Epstein, Millersville, MD (US); Luke Anthony Zanetti, Parkton, MD (US)

(21) Appl. No.: 18/328,608

(22) Filed: Jun. 2, 2023

Related U.S. Application Data
(63) Continuation of application No. PCT/US21/61621, filed on Dec. 2, 2021.
(60) Provisional application No. 63/121,233, filed on Dec. 3, 2020.

(51) Int. Cl. A61F 2/24

(52) U.S. Cl. CPC A61F 2/2466; A61F 2/2457; A61F 2220/0075; A61F 2220/0016; A61B 2017/0409

(57) ABSTRACT

A delivery system comprises two or more needles including at least a first needle comprising a distal tip configured to pierce a valve leaflet of a heart, two or more leaflet anchors, each of the two or more leaflet anchors configured to be delivered via one of the two or more needles, and a shaft comprising a first interior lumen configured to slidably receive the two or more needles, the shaft having a first opening into the first interior lumen at a distal end of the shaft, the first opening configured to allow at least the first needle to exit the shaft.

### CROSS REFERENCE TO RELATED APPLICATIONS

This application is a continuation of International Patent Application No. PCT/US2021/061621, filed Dec. 2, 2021, which claims the benefit of U.S. Patent Application No. 63/121,233, filed on Dec. 3, 2020, the entire disclosures all of which are incorporated by reference for all purposes.

### BACKGROUND

### Technical Field

The disclosure herein relates to cardiac valve repairs, and more particularly to surgical suture placement in connection with minimally invasive valve repair operations.

### Description of Related Art

Certain medical and other procedures involve the use of sutures or other similar devices.

### SUMMARY

For purposes of summarizing the disclosure, certain aspects, advantages and novel features have been described herein. It is to be understood that not necessarily all such advantages may be achieved in accordance with any particular instance. Thus, the disclosed instances may be carried out in a manner that achieves or optimizes one advantage or group of advantages as taught herein without necessarily achieving other advantages as may be taught or suggested herein.

Some implementations of the present disclosure relate to a delivery system comprising two or more needles including at least a first needle comprising a distal tip configured to pierce a valve leaflet of a heart and two or more leaflet anchors. Each of the two or more leaflet anchors is configured to be delivered via one of the two or more needles. The delivery system further comprises a shaft comprising a first interior lumen configured to slidably receive the two or more needles. The shaft has a first opening into the first interior lumen at a distal end of the shaft. The first opening is configured to allow at least the first needle to exit the shaft.

The first interior lumen may have a non-constant diameter that gradually decreases from a maximal diameter of the first interior lumen to a minimal diameter at the first opening that is less than the maximal diameter of the first interior lumen. In some instances, the first opening is configured to allow only one of the two or more needles to exit the shaft at a time.

In some instances, a first leaflet anchor of the two or more leaflet anchors is configured to be delivered via the first needle and a second leaflet anchor of the two or more leaflet anchors is configured to be delivered via a second needle of the two or more needles. Each of the two or more leaflet anchors may comprise a suture wrapped at least partially around a distal portion of one of the two or more needles and configured to form a knot anchor when removed from the two or more needles.

The delivery system may further comprise two or more pushers, each of the two or more pushers having interior lumens and configured to be disposed at least partially within the first interior lumen of the shaft, slidably receive one of the two or more needles, and press against one of the two or more leaflet anchors to remove the leaflet anchor from one of the two or more needles.

In some instances, the delivery system further comprises a plunger configured to move the two or more needles out of the shaft.

The first needle may comprise a second interior lumen and a second opening into the second interior lumen at the distal tip of the first needle, the second interior lumen configured to carry the two or more leaflet anchors.

In some instances, the first needle comprises an elongate aperture into the second interior lumen. The elongate aperture may extend along at least a portion of a first side of the first needle to the second opening and may be configured to allow one or more suture tails of the two or more leaflet anchors to exit the first needle via the elongate aperture.

The elongate aperture may be configured to prevent the two or more leaflet anchors from entirely exiting the first needle via the elongate aperture. In some instances, a second needle of the two or more needles comprises a third interior lumen configured to receive the one or more suture tails.

In some instances, the two or more leaflet anchors may be pre-formed suture knots. The two or more leaflet anchors may be configured to be situated in a single-file orientation within the first needle.

In accordance with some implementations of the present disclosure, a method comprises delivering a shaft carrying two or more needles to a first portion of a leaflet of a heart ventricle. The shaft comprises a first interior lumen having a non-constant diameter that gradually decreases near an opening into the first interior lumen at a distal end of the shaft to prevent multiple of the two or more needles from exiting the shaft simultaneously at the opening. The method further comprises deploying a first needle of the two or more needles through the leaflet. The first needle carries a first sutureform. The method further comprises extending a first pusher along the first needle to remove the first sutureform from the first needle and to cause the first sutureform to form a first suture knot, retracting the first needle into the first interior lumen, moving the shaft to a second portion of the leaflet and deploying a second needle of the two or more needles through the leaflet. The second needle carries a second sutureform. The method further comprises extending a second pusher along the second needle to remove the second sutureform from the second needle and to cause the second sutureform to form a second suture knot and retracting the second needle into the first interior lumen.

The first needle may be configured to carry the first sutureform at an exterior surface of the first needle.

In some instances, the first needle comprises a second interior lumen and is configured to carry the first sutureform within the second interior lumen. The first needle may comprise a longitudinal slot into the second interior lumen along at least a portion of a first side of the first needle.

Some implementations of the present disclosure relate to a method comprising delivering a shaft carrying two or more needles to a first portion of a leaflet of a heart ventricle. A first needle of the two or more needles carries two or more suture anchors within a first interior lumen of the first needle. The first needle comprises an elongate aperture extending along at least a portion of a first side of the first needle to an opening at a distal tip of the first needle. The elongate aperture is configured to allow one or more suture tails of the two or more suture anchors to exit the first interior lumen. A second needle of the two or more needles is configured to receive the one or more suture tails into a second interior lumen of the second needle. The method further comprises deploying the first needle through the leaflet, extending a pusher along the first needle to press against at least one of the two or more suture anchors to remove a first suture anchor of the two or more suture anchors from the first needle, retracting the first needle from the leaflet, moving the shaft to a second portion of the leaflet, deploying the first needle through the leaflet, extending the pusher along the first needle to press against at least one of the two or more suture anchors to remove a second suture anchor of the two or more suture anchors from the first needle, and retracting the first needle out of the leaflet.

In some instances, the shaft comprises a third interior lumen having a non-constant diameter that gradually decreases near a second opening into the third interior lumen at a distal end of the shaft to prevent multiple of the two or more needles from exiting the shaft simultaneously at the second opening.

The method may further comprise deploying a third needle of the two or more needles through the leaflet. The third needle may be configured to carry two or more suture anchors.

In accordance with one or more implementations of the present disclosure, a delivery system comprises two or more needles. Each of the two or more needles comprises a distal tip configured to pierce a valve leaflet of a heart. The delivery system further comprises two or more leaflet anchors. Each of the two or more leaflet anchors is configured to be delivered via one of the two or more needles. The delivery system further comprises a multi-lumen shaft comprising two or more interior lumens. Each of the two or more interior lumens is configured to slidably receive one of the two or more needles and one of the two or more leaflet anchors and prevent contact between the two or more needles.

In some instances, each of the two or more leaflet anchors comprises a suture wrapped at least partially around a distal portion of one of the two or more needles and configured to form a knot anchor when removed from the two or more needles.

The delivery system may further comprise two or more pushers. Each of the two or more pushers may have an interior lumen and configured to be disposed at least partially within one of the two or more interior lumens of the multi-lumen shaft, slidably receive one of the two or more needles, and/or press against one of the two or more leaflet anchors to remove the leaflet anchor from one of the two or more needles.

In some instances, at least one of the two or more leaflet anchors comprises a T-fastener, grappling hook, spike, umbrella structure, or pad. The multi-lumen shaft may be configured to deliver the two or more needles to different positions at the valve leaflet.

The multi-lumen shaft may be configured to be rotated to change a position of at least one of the two or more needles. In some instances, the two or more interior lumens of the multi-lumen shaft comprise three interior lumens.

The two or more needles may comprise three needles. In some instances, the two or more leaflet anchors comprise three leaflet anchors.

In some instances, the delivery system further comprises a plunger configured to move the two or more needles out of the multi-lumen shaft. The plunger may be configured to move the two or more needles simultaneously.

The plunger may be configured to move the two or more needles sequentially.

In some instances, the delivery system further comprises a plunger track housing configured to receive the plunger therein at least in part. The multi-lumen shaft may be configured to be coupled to the plunger track housing.

A first needle of the two or more needles may be longer than a second needle of the two or more needles.

In some instances, the multi-lumen shaft further comprises an atraumatic tip configured to contact the valve leaflet. At least one of the two or more needles may be at least partially composed of a shape-memory material.

Some implementations of the present disclosure relate to a method comprising delivering a multi-lumen shaft comprising two or more interior lumens to a valve leaflet of a heart, deploying a first needle and a first leaflet anchor via a first lumen of the multi-lumen shaft and through a first point of the valve leaflet, rotating the multi-lumen shaft, and deploying a second needle and a second leaflet anchor via a second lumen of the multi-lumen shaft and through a second point of the valve leaflet. A first distance between the first point and a leaflet edge of the valve leaflet is greater than a second distance between the second point and the leaflet edge.

In some instances, the method further comprises deploying a third needle and a third leaflet anchor via a third lumen of the multi-lumen shaft and through a third point of the valve leaflet.

The first leaflet anchor may comprise a first set of two suture tails and the second leaflet anchor comprises a second set of two suture tails. In some instances, the method further comprises securing the first set of two suture tails and the second set of two suture tails to a ventricle wall.

In some instances, securing the first set of two suture tails involves passing the first set of two suture tails through a pledget and forming a first knot at the pledget from the first set of two suture tails.

The first needle may be longer than the second needle. In some instances, the first needle is configured to extend further out of the multi-lumen shaft than the second needle.

Each method disclosed herein also encompass one or more simulations of the method, which are useful, for example, for teaching, demonstration, testing, device development, and procedure development. For example, methods for treating or diagnosing a patient include corresponding simulated methods performed on a simulated patient. Suitable simulated patients or anthropogenic ghosts can include any combination of physical and virtual elements. Examples of physical elements include whole human or animal cadavers, or any portion thereof, including, organ systems, individual organs, or tissue; and manufactured cadaver, organ system, organ, or tissue simulations. Examples of virtual elements include visual simulations, which can be displayed on a screen; projected on a screen, surface, space, or volume; and holographic images. The simulation can also include one or more of another type of sensory input, for example, auditory, tactile, and olfactory stimuli.

### BRIEF DESCRIPTION OF THE DRAWINGS

Various instances are depicted in the accompanying drawings for illustrative purposes and should in no way be interpreted as limiting the scope of the inventions. In addition, various features of different disclosed instances can be combined to form additional instances, which are part of this disclosure. Throughout the drawings, reference numbers may be reused to indicate correspondence between reference elements. However, it should be understood that the use of similar reference numbers in connection with multiple drawings does not necessarily imply similarity between respective instances associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some instances or configurations.

FIG. 1 is a cutaway view of the human heart.

FIG. 2 is a perspective view of a tissue anchor delivery device in accordance with one or more examples.

FIG. 3 is a cutaway view of a tissue anchor delivery device disposed at least partially within a chamber of a heart in accordance with one or more examples.

FIG. 4 is a perspective view of a dilator and introducer that may be used in valve-repair procedures in accordance with one or more examples.

FIG. 5 shows the introducer device and a tissue anchor delivery device shaft in accordance with one or more instances in accordance with one or more examples.

FIG. 6 is a close-up view of a distal portion of a tissue anchor delivery device shaft assembly positioned against a target heart valve leaflet in accordance with one or more examples.

FIG. 7 shows a cutaway view of a sutureform deployed from the anchor delivery device in a heart in accordance with one or more examples.

FIG. 8 shows a cutaway view of a deployed suture anchor a heart in accordance with one or more examples.

FIG. 9 shows a plurality of suture pairs passing through a cardiac tissue wall and through a pledget in accordance with one or more examples.

FIG. 10A provides a perspective view of a distal portion of a multi-lumen medical device shaft in accordance with one or more examples.

FIG. 10B is a perspective view of a distal portion of a multi-lumen medical device shaft having multiple needles and sutureforms projected from the distal portion of the shaft 110 in accordance with one or more examples.

FIG. 11A provides a perspective view of a distal portion of another medical device shaft configured for delivery of multiple delivery devices (e.g., needles) and/or implants (e.g., suture anchors) in accordance with one or more examples in accordance with one or more examples.

FIG. 11B is a perspective view of a distal portion of a multi-needle medical device shaft having a needle and sutureform projected from the distal portion of the shaft in accordance with one or more instances.

FIG. 12 illustrates a multi-lumen shaft for delivering multiple needles to a valve leaflet

FIG. 13 illustrates a single-lumen shaft for delivering multiple needles to a valve leaflet in accordance with one or more examples.

FIGS. 14A and 14B illustrate interior views of a delivery device that can be used to deliver and/or form one or more leaflet anchors to be disposed on a distal side of a mitral valve leaflet in accordance with some examples.

FIG. 15 (FIGS. 15-1 and 15-2) is a flow diagram illustrating a process for delivering multiple leaflet anchors to a valve leaflet in accordance with one or more instances of the present disclosure.

FIG. 16 (FIGS. 16-1 and 16-2) provides illustrations corresponding to steps of the process described in FIG. 15.

FIG. 17 illustrates an example delivery system for one or more suture anchors 190 in accordance with one or more instances of the present disclosure

FIG. 18 illustrates another example delivery system for one or more suture anchors 190 in accordance with one or more instances of the present disclosure.

FIG. 19 (FIGS. 19-1, 19-2, and 19-3) is a flow diagram illustrating a process for delivering multiple leaflet anchors to a valve leaflet in accordance with one or more instances of the present disclosure.

FIG. 20 (FIGS. 20-1, 20-2, and 20-3) provides illustrations corresponding to steps of the process described in FIG. 19.

FIG. 21A illustrates a multi-lumen shaft delivering multiple needles to a valve leaflet in accordance with one or more examples.

FIG. 21B illustrates rotation of the multi-lumen shaft to facilitate anchoring at or near the leaflet edge in accordance with one or more examples.

FIG. 22 (FIGS. 22-1 and 22-2) is a flow diagram illustrating a process for delivering multiple leaflet anchors to a valve leaflet in accordance with one or more instances of the present disclosure.

FIG. 23 (FIGS. 23-1 and 23-2) provides illustrations corresponding to steps of the process described in FIG. 22.

To further clarify various aspects of instances of the present disclosure, a more particular description of certain instances will be made by reference to various aspects of the appended drawings. It is appreciated that these drawings depict only typical instances of the present disclosure and are therefore not to be considered limiting of the scope of the disclosure. Moreover, while the figures can be drawn to scale for some instances, the figures are not necessarily drawn to scale for all instances. Instances of the present disclosure will be described and explained with additional specificity and detail through the use of the accompanying drawings.

### DETAILED DESCRIPTION

The headings provided herein are for convenience only and do not necessarily affect the scope or meaning of the claimed invention.

Although certain preferred instances and examples are disclosed below, inventive subject matter extends beyond the specifically disclosed instances to other alternative instances and/or uses and to modifications and equivalents thereof. Thus, the scope of the claims that may arise herefrom is not limited by any of the particular instances described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain instances; however, the order of description should not be construed to imply that these operations are order dependent. Additionally, the structures, systems, and/or devices described herein may be embodied as integrated components or as separate components. For purposes of comparing various instances, certain aspects and advantages of these instances are described. Not necessarily all such aspects or advantages are achieved by any particular instance. Thus, for example, various instances may be carried out in a manner that achieves or optimizes one advantage or group of advantages as taught herein without necessarily achieving other aspects or advantages as may also be taught or suggested herein.

The following description refers to the accompanying drawings, which illustrate specific instances. Other instances having different structures and operation do not depart from the scope of the disclosure.

Instances of the present disclosure provide solutions relating to the treatment of certain structural heart conditions with multiple valve leaflet anchors and/or devices configured to deliver multiple valve leaflet anchors. Various disease processes can impair the proper functioning of one or more of the valves of the heart. These disease processes include degenerative processes (e.g., Barlow's disease, fibroelastic deficiency), inflammatory processes (e.g., rheumatic heart disease), and infectious processes (e.g., endocarditis). Additionally, damage to the ventricle from prior heart attacks (e.g., myocardial infarction secondary to coronary artery disease) or other heart diseases (e.g., cardiomyopathy) can distort the geometry of the heart causing valves in the heart to dysfunction. Many patients undergoing valve surgery, such as mitral valve surgery, suffer from a degenerative disease that causes a malfunction in a leaflet of the valve, which results in prolapse and regurgitation.

Valve regurgitation occurs when the leaflets of the valve do not close completely, thereby allowing blood to leak back into the prior chamber when the heart contracts. There are generally three mechanisms by which a valve becomes regurgitant or incompetent, including Carpentier's type I, type II and type III malfunctions. A Carpentier type I malfunction involves the dilation of the annulus such that the area of the valve orifice increases. The otherwise normally functioning leaflets do not have enough surface area to cover the enlarged orifice and fail to form a tight seal (e.g., do not coapt properly) causing regurgitation. Included in a type I mechanism malfunction are perforations of the valve leaflets, as in endocarditis. A Carpentier's type II malfunction involves prolapse of a segment of one or both leaflets above the plane of coaptation. This is the most commonly treated cause of mitral regurgitation, and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic heart disease (IIIa) or dilation of the ventricle (IIIb).

Mitral valve disease is the most common valvular heart disorder, with nearly 4 million Americans estimated to have moderate to severe mitral valve regurgitation (“MR”), with similar numbers of individuals impacted outside of the United States. MR can result in a volume overload on the left ventricle which in turn progresses to ventricular dilation, decreased ejection performance, pulmonary hypertension, symptomatic congestive heart failure, atrial fibrillation, right ventricular dysfunction, and/or death. Successful surgical mitral valve repair can at least partially restore mitral valve competence, abolish the volume overload on the left ventricle, improve symptom status, and/or prevent adverse left ventricular remodeling. While generally safe and effective, conventional open-heart operations are invasive, result in significant disability, and require extended post-procedure recovery. Patients routinely spend five to seven days in the hospital and often are not able to return to normal daily activities for a month or more.

In many instances of mitral valve regurgitation, repair may be preferable to valve replacement. There are a variety of advantages to performing heart valve repair (e.g., mitral valve repair) using less invasive procedures while the heart is still beating, as described in detail herein. Mitral valve repair procedures may rely upon use of visualization technology, such as sonic guidance, which may have limitations that can reduce the effectiveness of such repairs. Accordingly, there is a continuing need for new procedures and devices for performing less invasive mitral valve repairs which do not require cardiac arrest and are less technologically challenging.

In some implementations, the present disclosure relates to an anchor delivery system configured to deliver multiple suture knots simultaneously, near-simultaneously, and/or during a single deployment stage. The term “suture” is used herein according to its plain and ordinary meaning and may refer to any elongate cord strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord” and “suture” may be used substantially interchangeably. In addition, use of the singular form of any of the suture-related terms listed above, including the terms “suture” and “cord,” may be used to refer to a single suture/cord, or to a portion thereof. For example, where a suture knot or anchor is deployed on a distal side of a tissue portion, and where two suture portions extend from the knot/anchor on a proximal side of the tissue, either of the suture portions may be referred to as a “suture” or a “cord,” regardless of whether both portions are part of a unitary suture or cord.

While suture knots may be implanted one at a time, it may require an average of approximately seven minutes to navigate a delivery device to a valve leaflet and/or deploy a single suture knot. Accordingly, the time and/or level of invasiveness required to deploy multiple leaflets may be multiplied for each additional suture knot delivered. It is therefore advantageous to deliver and/or deploy multiple (e.g., three) suture knots during a single stage and/or by navigating a delivery device to the valve leaflet only once.

In some instances, a delivery device may comprise a shaft having multiple (e.g., three) lumens configured to each receive a needle carrying a suture. Such a delivery device may significantly reduce the required procedure time and/or increase the ease of use for delivering multiple suture knots. A needle may be composed at least partially of metal and/or other suitable material. In some instances, a needle may be at least partially composed of a shape-memory material/alloy (e.g., nitinol) and/or may be shaped to increase separation between multiple needles after the needles exit the shaft.

Moreover, such a delivery device may advantageously simplify delivery one or more suture knots at or near an edge portion of a valve leaflet. For example, the difficulty of piercing and/or delivering a suture knot to a valve leaflet may increase towards the edge portion of the valve leaflet. In some instances, a delivery device may allow for sequential delivery of the multiple needles via the device chamber to prevent slippage of the needles and/or suture knots delivered via the needles.

In some instances, each needle passed and/or slidably received through an interior lumen of a multi-lumen chamber may be configured to carry and/or deliver at least one suture and/or suture knot to a leaflet of a heart valve. Each needle may further be configured to puncture a valve leaflet and/or deploy a suture knot on a distal (e.g., atrial) side of the leaflet. The needles and/or suture knots may be deployed sequentially and/or simultaneously. Each of the needles may be configured to puncture and/or pass through a separate hole in the valve leaflet. The interior lumens of the multi-lumen chamber may be separated by a desired and/or appropriate distance to ensure that multiple needles do not pass through a single hole in the leaflet.

Certain inventive features disclosed herein relate to the delivery of suture knots associated with certain heart valve repair systems and devices, and/or systems, process, and devices for repairing any other type of target organ tissue. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, or otherwise physically related to the second feature, element, component, device, or member.

The following includes a general description of human cardiac anatomy that is relevant to certain inventive features and instances disclosed herein and is included to provide context for certain aspects of the present disclosure. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary trunk, aorta, etc.).

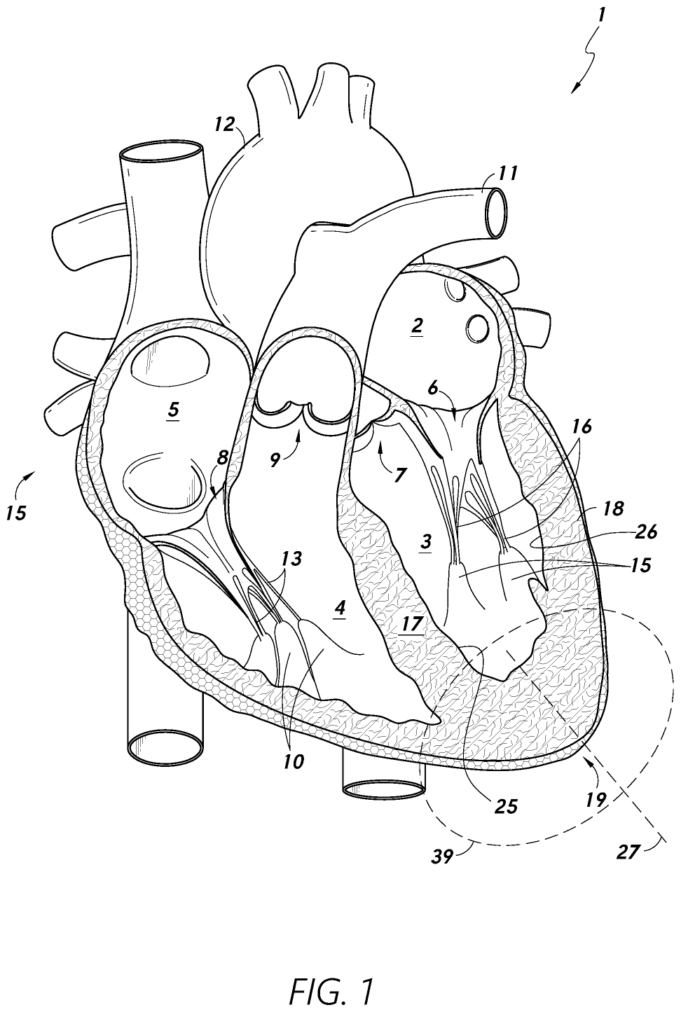

FIG. 1 illustrates an example representation of a heart 1 having various features relevant to certain aspects of the present inventive disclosure. The heart 1 includes four chambers, namely the left ventricle 3, the left atrium 2, the right ventricle 4, and the right atrium 5. A wall of muscle, referred to as the septum 17, separates the left 2 and right 5 atria and the left 3 and right 4 ventricles. The inferior tip 19 of the heart 1 is referred to as the “apex” 19 and is generally located on the midclavicular line, in the fifth intercostal space. The apex 19 can be considered part of the greater apical region 39.

The left ventricle 3 is the primary pumping chamber of the heart 1. A healthy left ventricle is generally conical and/or apical in shape in that it is longer (along a longitudinal axis extending in a direction from the aortic valve 7 to the apex 19) than it is wide (along a transverse axis extending between opposing walls 25, 26 at the widest point of the left ventricle) and descends from a base (including the left ventricle papillary muscles 15) with a decreasing cross-sectional circumference to the point or apex 19. Generally, the apical region 39 of the heart is a bottom region of the heart that is within the left or right ventricular region but is distal to the mitral 6 and tricuspid 8 valves and toward the tip of the heart. More specifically, the apical region 39 may be considered to be within about 20 cm to the right or to the left of the median axis 27 of the heart 1.

The pumping of blood from the left ventricle is accomplished by a squeezing motion and a twisting or torsional motion. The squeezing motion occurs between the lateral wall 18 of the left ventricle and the septum 17. The twisting motion is a result of heart muscle fibers that extend in a circular or spiral direction around the heart. When these fibers contract, they produce a gradient of angular displacements of the myocardium from the apex 19 to the base about the longitudinal axis of the heart. The resultant force vectors extend at angles from about 30-60 degrees to the flow of blood through the aortic valve 7. The contraction of the heart is manifested as a counterclockwise rotation of the apex 19 relative to the base, when viewed from the apex 19. A healthy heart can pump blood from the left ventricle in a very efficient manner due to the spiral contractility of the heart.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (e.g., mitral and tricuspid) heart valves may comprise a collection of chordae tendineae (13, 16) and papillary muscles (10, 15) for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10.

Surrounding the ventricles (3, 4) are a number of arteries (not shown) that supply oxygenated blood to the heart muscle and a number of veins that return the blood from the heart muscle. The coronary sinus (not shown) is a relatively large vein that extends generally around the upper portion of the left ventricle 3 and provides a return conduit for blood returning to the right atrium 5. The coronary sinus terminates at the coronary ostium (not shown) through which the blood enters the right atrium.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

Various disease processes can impair the proper functioning of one or more of the valves of the heart. These disease processes include degenerative processes (e.g., Barlow's disease, fibroelastic deficiency), inflammatory processes (e.g., rheumatic heart disease) and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in one or more leaflets of the valve which results in prolapse and regurgitation.

The mitral valve 6 and tricuspid valve 8 can be divided into three parts: an annulus, leaflets, and a sub-valvular apparatus. The sub-valvular apparatus can be considered to include the papillary muscles 10, 15 and the chordae tendineae 13, 16, which can elongate and/or rupture. If a valve is functioning properly, when closed, the free margins or edges of the leaflets come together and form a tight junction, the are of which, in the mitral valve, is known as the line, plane or area of coaptation. Normal mitral and tricuspid valves open when the ventricles relax allowing blood from the atrium to fill the decompressed ventricle. When the ventricle contracts, the chordae tendineae advantageously properly tether or position the valve leaflets such that the increase in pressure within the ventricle causes the valve to close, thereby preventing blood from leaking into the atrium and assuring that substantially all of the blood leaving the ventricle is ejected through the aortic valve 7 or pulmonic valve 9 and into the arteries of the body. Accordingly, proper function of the valves depends on a complex interplay between the annulus, leaflets, and sub-valvular apparatus. Lesions in any of these components can cause the valve to dysfunction and thereby lead to valve regurgitation.

Generally, there are three mechanisms by which a heart valve becomes regurgitant or incompetent; they include Carpentier's type I, type II and type III malfunctions. A Carpentier type I malfunction involves the dilation of the annulus such that normally functioning leaflets are distracted from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

One or more chambers in the heart 1 may be accessed in accordance with certain heart valve-repair procedures and/or other interventions. Access into a chamber in the heart may be made at any suitable site of entry. In some implementations, access is made to a chamber of the heart, such as a target ventricle (e.g., left ventricle) associated with a diseased heart valve, through the apical region 39. For example, access into the left ventricle 3 (e.g., to perform a mitral valve repair) may be gained by making a relatively small incision at the apical region 39, close to (or slightly skewed toward the left of) the median axis 27 of the heart. Access into the right ventricle 4 (e.g., to perform a tricuspid valve repair) may be gained by making a small incision into the apical region 39, close to or slightly skewed toward the right of the median axis 27 of the heart. Accordingly, the ventricle can be accessed directly via the apex, or via an off-apex location that is in the apical region 39 but slightly removed from the tip/apex, such as via lateral ventricular wall, a region between the apex and the base of a papillary muscle, or even directly at the base of a papillary muscle. In some implementations, the incision made to access the appropriate ventricle of the heart is no longer than about 1 mm to about 5 cm, from about 2.5 mm to about 2.5 cm, or from about 5 mm to about 1 cm in length. When a percutaneous approach is sought, no incision into the apex region of the heart may be made, but rather access into the apical region 39 may be gained by direct needle puncture, for instance by an 18-gauge needle, through which an appropriate repair instrument can be advanced.

Certain inventive features disclosed herein relate to certain heart valve repair systems and devices, and/or systems, process, and devices for repairing any other type of target organ tissue. In some implementations, a tissue anchor delivery device may be employed in repairing a mitral valve in patients suffering from degenerative mitral regurgitation or other condition. In some implementations, a transapical off-pump echo-guided repair procedure is implemented in which at least part (e.g., a shaft portion/assembly) of a valve repair system is inserted in the left ventricle and steered to the surface of the diseased portion of a target mitral valve leaflet and used to deploy/implant a tissue anchor in the target leaflet. The tissue anchor (e.g., sutureform formed into a bulky knot, T-fastener, grappling hook, spike, umbrella structure, pad) may advantageously be integrated or coupled with one or more artificial/synthetic cords serving a function similar to that of chordae tendineae. Such artificial cord(s) may comprise suture(s) and/or suture ends and/or tail portions associated with a knot-type tissue anchor and may comprise any suitable or desirable material, such as expanded polytetrafluoroethylene (ePTFE) or the like. In some instances, cords and/or anchors described herein may be at least partially composed of organic, polymer, metal, and/or composite materials and/or may be at least partially ceramic-based.

Processes for repairing a target organ tissue, such as repair of mitral valve leaflets to address mitral valve regurgitation, can include inserting a tissue anchor delivery device, such as a delivery device as described in PCT Application No. PCT/US2012/043761, (published as WO 2013/003228, and referred to herein as “the '761 PCT Application”) and/or in PCT Application No. PCT/US2016/055170 (published as WO 2017/059426 and referred to herein as “the '170 PCT Application”), the entire disclosures of which are incorporated herein by reference, into a body and extending a distal end of the delivery device to a proximal side of the target tissue (e.g., leaflet).

The '761 PCT Application and the '170 PCT Application describe in detail methods and devices for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which may depend on the specific abnormality and the tissues involved.

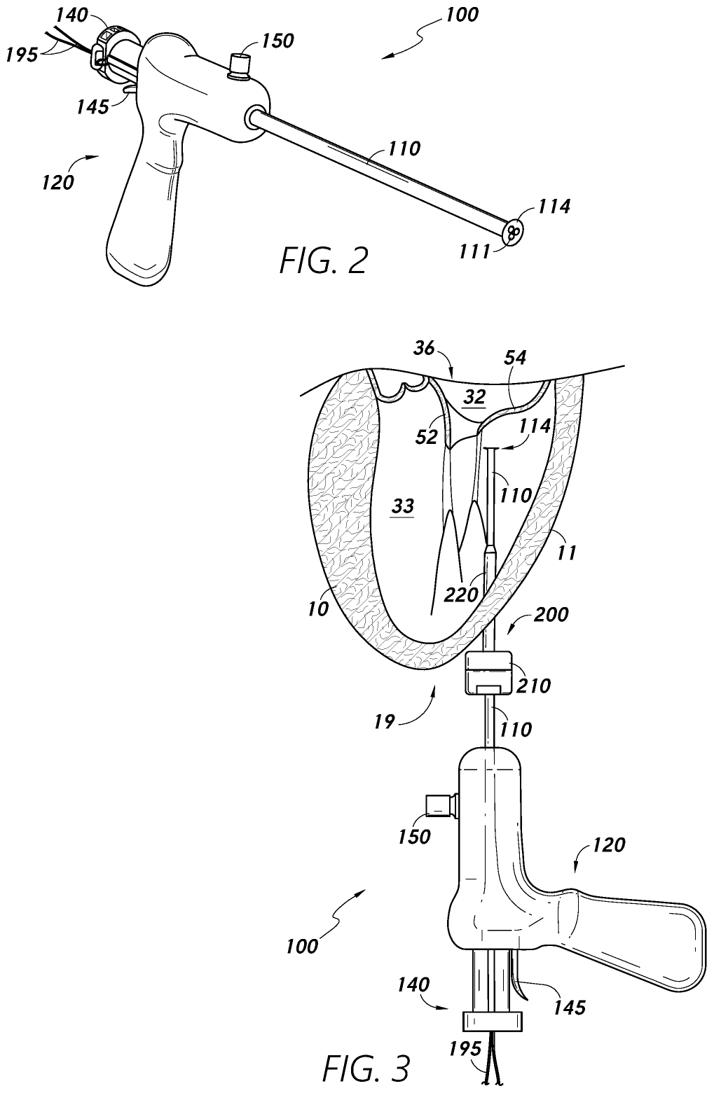

FIG. 2 is a perspective view of a tissue anchor delivery system 100 in accordance with one or more instances. The tissue anchor delivery system 100 may be used to repair a heart valve, such as a mitral valve, and improve functionality thereof. For example, the tissue anchor delivery system 100 may be used to reduce the degree of mitral regurgitation in patients suffering from mitral regurgitation caused by, for example, midsegment prolapse of valve leaflets as a result of degenerative mitral valve disease. In order to repair such a valve, the tissue anchor delivery system 100 may be utilized to deliver and anchor tissue anchors, such as suture-knot-type tissue anchors, in a prolapsed valve leaflet. As described in detail below, such procedures may be implemented on a beating heart.

The delivery system 100 includes a rigid elongate tube 110 forming at least one internal working lumen 111. In some instances, the elongate tube 110 may comprise multiple (e.g., three) lumens 111 and/or one or more lumens 111 having non-constant diameters. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure. Although described in certain instances and/or contexts as comprising a rigid elongate tube, it should be understood that tubes, shafts, lumens, conduits, and the like disclosed herein may be either rigid, at least partially rigid, flexible, and/or at least partially flexible. Therefore, any such component described herein, whether or not referred to as rigid herein should be interpreted as possibly being at least partially flexible. In accordance with the present disclosure, the rigid elongate tube 110 may be referred to as a “shaft” for simplicity. Implementation of a valve-repair procedure utilizing the delivery system 100 can be performed in conjunction with certain imaging technology designed to provide visibility of the shaft 110 of the delivery system 100 according to a certain imaging modality, such as echo imaging. Generally, when performing a valve-repair procedure utilizing the tissue anchor delivery system 100, the operating physician may advantageously work in concert with an imaging technician, who may coordinate with the physician to facilitate successful execution of the valve-repair procedure.

In addition to the delivery shaft 110, the delivery system 100 may include a plunger feature 140, which may be used or actuated to manually deploy a leaflet anchor, which may include a pre-formed knot, such as a bulky knot as described in detail below. The tissue anchor delivery system 100 may further include a plunger lock mechanism 145, which may serve as a safety lock that locks the valve delivery system until ready for use or deployment of a leaflet anchor as described herein. The plunger 140 may have associated therewith a suture-release mechanism, which may be configured to lock in relative position one or more pairs of suture ends/tails 195 associated with a pre-formed knot anchor (not shown) to be deployed. For example, the suture portions 195 may be ePTFE sutures. In some instances, the suture-release mechanism may be configured to lock more than two suture tails 195. For example, the shaft 110 may be configured to receive three suture knots, with each suture knot having two suture tails 195. Accordingly, the suture-release mechanism may be configured to lock six suture tails 195. The system 100 may further comprise a flush port 150, which may be used to de-air the lumen of the shaft 110. For example, heparinized saline flush, or the like, may be connected to the flush port 150 using a female Luer fitting to de-air the valve repair system 100.

The one or more lumens 111 of the shaft 110 may be configured to house one or more needles (not shown) that may be configured to be wrapped at least in part with one or more pre-formed knot sutureform anchors, as described in detail herein. In some instances, the shaft 110 may be configured to form a relatively low profile. For example, the shaft 110 may have a diameter of approximately 10 mm (about 0.5 inch) less. The shaft 110 may be associated with an atraumatic tip 114 feature. The atraumatic tip 114 can be an echogenic leaflet-positioner component, which may be used for deployment and/or positioning of the suture-type tissue anchor. In some instances, the tip 114 may be cone-shaped and/or may have any other shape. The atraumatic tip 114, disposed at the distal end of the shaft 110, may be configured to have deployed therefrom one or more wrapped pre-formed suture knots (e.g., sutureforms), as described herein.

The atraumatic tip 114 may be referred to as an “end effector.” In addition to one or more pre-formed knot sutureforms and/or associated needles, the shaft 110 may be configured to house one or more elongated knot pusher tubes (not shown; also referred to herein as “pushers”), which may be actuated simultaneously or sequentially using the plunger 140 in some instances. As described in further detail below, the tip 114 can provide a surface against which the target valve leaflet may be held in connection with deployment of a leaflet anchor.

The delivery device 100 may be used to deliver one or more tissue anchors, as described in greater detail below. For example, the delivery device 100 may be utilized to deliver one or more tissue anchors (e.g., bulky knots) on a distal side of a mitral valve leaflet. While some instances described herein may refer to and/or describe “bulky knot” type tissue anchors, the delivery systems described herein may be configured for delivery of any of a variety of anchor types, including T-fasteners, hooks (e.g., grappling hooks), spikes, umbrella- and/or disc-shaped structures, and/or pads. The tip 114 (e.g., end effector), can be placed in contact with the ventricular side of a leaflet of a mitral valve. The tip 114 can be coupled to the distal end portion of the shaft 110, wherein the proximal end portion of the shaft 110 may be coupled to a handle portion 120 of the delivery device 100, as shown. Generally, the one or more elongate pushers (not shown) may be configured to be movably disposed within the one or more lumens 111 of the shaft 110 and/or coupled to a pusher hub (not shown) that may be movably disposed within the handle 120 and/or releasably coupled to the plunger 140. One or more needles (not shown) carrying one or more pre-formed tissue anchor sutureforms can be movably disposed within lumens of the one or more pushers and/or coupled to a needle hub (not shown) that may be coupled to the plunger 140. The plunger 140 can be used to actuate or move the one or more needles and/or pushers during deployment of one or more distal anchors and/or may be movably disposed at least partially within the handle 120. For example, the handle 120 may define one or more lumens in which the plunger 140 can be moved. During operation, each of the one or more pushers may also move within one or more lumens of the handle 120. The plunger lock 145 can be used to prevent the plunger 140 from moving within the handle 120 during storage and prior to performing a procedure to deploy one or more tissue anchors.

One or more needles may have a pre-formed knot disposed about a distal portion thereof while maintained in the shaft 110. For example, a pre-formed knot may be formed of one or more sutures configured in a coiled sutureform having a plurality of winds/turns around a needle over a portion of the needle that may be associated with a longitudinal slot in the needle that can run from the distal end thereof. Although the term “sutureform” is used herein, it should be understood that such components/forms may comprise suture, wire, and/or any other elongate material wrapped or formed in a desired configuration. Moreover, the term “sutureform” may be used generally interchangeably herein with the terms “leaflet anchors” and/or “suture anchors” in some contexts. The coiled sutureform can be provided or shipped disposed around the needle. In some instances, one or more suture tails 195 (e.g., two per needle) may extend from the coiled sutureform. The suture tails 195 may extend through the lumen of the needle and/or through a passageway of the plunger 140 and may exit the plunger 140 at a proximal end portion thereof. The coiled sutureform may advantageously be configured to be formed into a suture-type tissue anchor (referred to herein as a “bulky knot”) in connection with an anchor-deployment procedure, as described in more detail below. The coiled sutureform can be configurable to a knot/deployed configuration by approximating opposite ends of the coiled portion thereof towards each other to form one or more loops.

The delivery device 100 can further include a suture/tether catch mechanism (not shown) coupled to the plunger 140 at a proximal end of the delivery device 100, which may be configured to releasably hold or secure one or more sutures extending through the delivery device 100 during delivery of a tissue anchor as described herein. The suture catch can be used to hold the suture(s) with a friction fit and/or with a clamping force and/or can have a lock that can be released after the tissue anchor has been deployed/formed into a bulky knot, as described herein.

FIG. 3 is a cutaway view of a tissue anchor delivery device 100 disposed at least partially within a chamber of a heart in accordance with one or more instances. As described herein, the anchor delivery device 100 can be used in beating heart mitral valve repair procedures. In some instances, the shaft 110 of the delivery device 100 can be configured to extend and/or contract with the beating of the heart. During systolic contraction, the median axis of the heart generally shortens. For example, the distance from the apex 19 of the heart to the valve leaflets 52, 54 can vary by about 1 centimeter (cm) to about 2 cms with each heartbeat in some patients. In some instances, the length of the shaft 110 that protrudes from the handle 120 can change with the length of the median axis of the heart. That is, distal end of the shaft 110 can be configured to be floating such that the shaft can extend and retract with the beat of the heart so as to maintain contact with the target mitral valve leaflet.

Advancement of the delivery device 100 may be performed in conjunction with echo imaging, direct visualization (e.g., direct transblood visualization), and/or any other suitable remote visualization technique/modality. With respect to cardiac procedures, for example, the delivery device 100 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). Typical procedures that can be implemented using echo guidance are set forth in Suematsu, Y., J. Thorac. Cardiovasc. Surg. 2005; 130:1348-56 (“Suematsu”), the entire disclosure of which is incorporated herein by reference.

According to some implementations of valve-repair procedures, an incision into the apical region 19 of the appropriate ventricle 33 of the heart is made. For instance, an introducer port device 200 containing a one or more fluid-retention valves to prevent blood loss and/or air entry into the ventricle 33, may be inserted into the site of entry. Once inside the chamber 33, the shaft 110 of the delivery device 100 may be advanced through the lumen 220 of the introducer 200. In some instances, a sheath may be inserted through the introducer 200, through which one or more other instruments are advanced. For instance, an endoscope may first be advanced into the chamber 33 to visualize the ventricle, the valve 36, and/or the sub-valvular apparatus. By use of an appropriate endoscope, a careful analysis of the malfunctioning valve 36 may be performed. Each segment of each leaflet may be carefully assessed to determine its pliability, integrity, and motion. Based on this assessment, the practitioner can determine whether the valve can indeed be repaired or must be replaced. The motion of the leaflets 52, 54 can be classified as slightly dysfunctional, prolapsed, or restricted and based on this classification, the necessary steps of the repair can be determined.

Mitral valve regurgitation generally increases the workload on the heart and may lead to very serious conditions if left untreated, such as decreased ventricular function, pulmonary hypertension, congestive heart failure, permanent heart damage, cardiac arrest, and ultimately death. Since the left heart is primarily responsible for circulating the flow of blood throughout the body, malfunction of the mitral valve 36 is particularly problematic and often life threatening. Methods and devices are provided herein, as well as in the '761 PCT Application and the '170 PCT Application, for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which will depend on the specific abnormality and the tissues involved.

After a minimally invasive approach is determined to be advisable, one or more incisions may be made proximate to the thoracic cavity to provide a surgical field of access. The total number and length of the incisions to be made depend on the number and types of the instruments to be used as well as the procedure(s) to be performed. The incision(s) may advantageously be made in such a manner as to be minimally invasive. As referred to herein, the term “minimally invasive” means in a manner by which an interior organ or tissue may be accessed with relatively little damage being done to the anatomical structure through which entry is sought. For example, a minimally invasive procedure may involve accessing a body cavity by a small incision of, for example, approximately 5 cm or less made in the skin of the body. The incision may be vertical, horizontal, or slightly curved. If the incision is located along one or more ribs, it may advantageously follow the outline of the rib. The opening may advantageously extend deep enough to allow access to the thoracic cavity between the ribs or under the sternum and is preferably set close to the rib cage and/or diaphragm, dependent on the entry point chosen.

In one example method, the heart may be accessed through one or more openings made by one or more small incision in a portion of the body proximal to the thoracic cavity, such as between one or more of the ribs of the rib cage of a patient, proximate to the xyphoid appendage, or via the abdomen and diaphragm. Access to the thoracic cavity may be sought to allow the insertion and use of one or more thorascopic instruments, while access to the abdomen may be sought to allow the insertion and use of one or more laparoscopic instruments. Insertion of one or more visualizing instruments may then be followed by transdiaphragmatic access to the heart. Additionally, access to the heart may be gained by direct puncture (e.g., via an appropriately sized needle, for instance an 18-gauge needle) of the heart from the xyphoid region. Accordingly, the one or more incisions should be made in such a manner as to provide an appropriate surgical field and access site to the heart in the least invasive manner possible. Access may also be achieved using percutaneous methods, further reducing the invasiveness of the procedure. See, e.g., “Full-Spectrum Cardiac Surgery Through a Minimal Incision Mini-Sternotomy (Lower Half) Technique,” Doty et al., Annals of Thoracic Surgery 1998; 65(2): 573-7 and “Transxiphoid Approach Without Median Sternotomy for the Repair of Atrial Septal Defects,” Barbero-Marcial et al., Annals of Thoracic Surgery 1998; 65(3): 771-4, the entire disclosures of each of which are incorporated herein by reference.

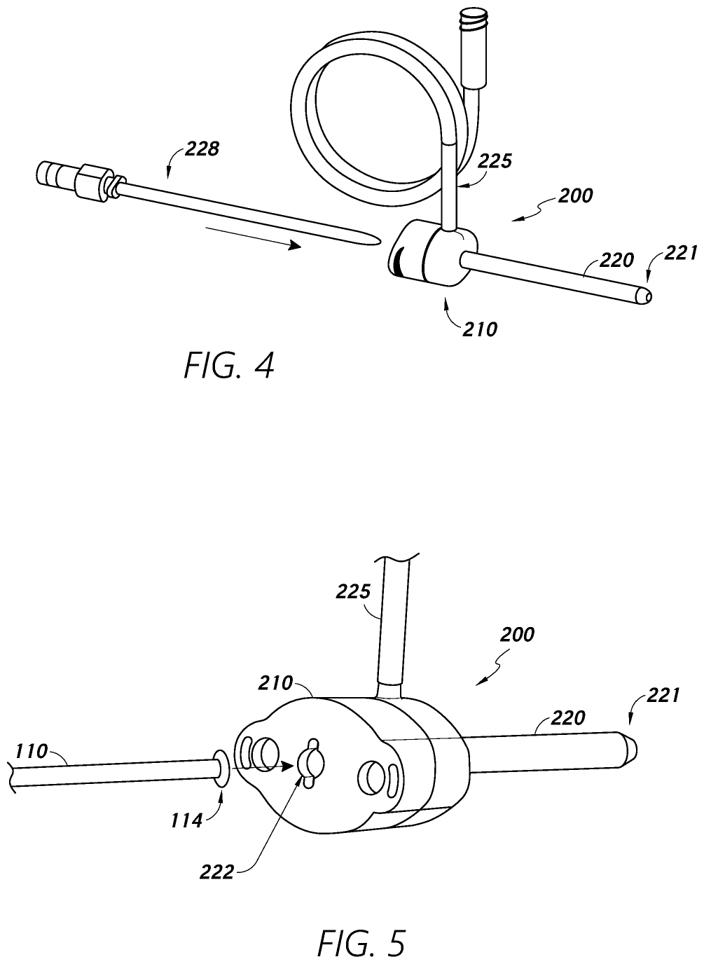

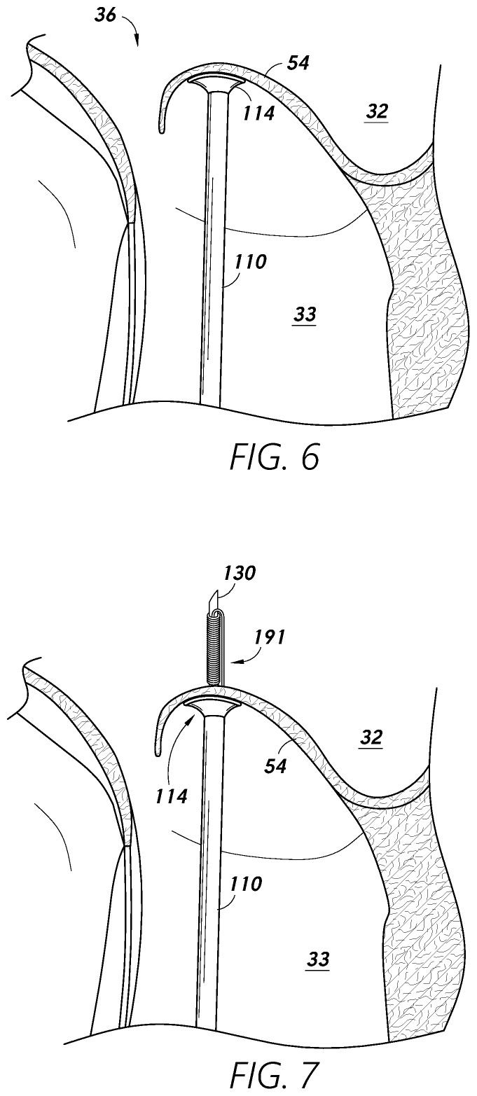

FIG. 4 is a perspective view of a dilator 228 and introducer 200 that may be used in valve-repair procedures in accordance with one or more instances. FIG. 5 shows the introducer device 200 and a tissue anchor delivery device shaft 110 in accordance with one or more instances.

The hemostatic introducer 200 may be inserted into the target ventricle at a tip 221 associated with a lumen 220 of the introducer 200. The lumen 220 of the introducer 200 may be used to guide the shaft 110 of a tissue anchor delivery device in accordance with instances of the present disclosure during a valve-repair procedure. The body or hub 210 of the introducer 200 may be used to secure the introducer 200 to the pericardium of the heart for stable entry of the shaft 110 of the tissue anchor delivery device and/or to control the amount of bleed-back during the valve-repair procedure. In some instances, a female Luer may be used to de-air the introducer 200 through a port 225 prior to use and/or to connect a fluid flush, such as a heparin flush, during a valve-repair procedure. The dilator 228 may be used to guide the introducer into the target ventricle. For example, the dilator 228 may be used to guide the introducer 200 into the left ventricle off-apex, as described in detail herein. In some implementations, a tie-down eyelet is used to secure the introducer 200 during the valve-repair procedure.

The introducer lumen 220 provides a conduit into a target surgical area or chamber, such as a ventricle of a heart. In some instances, the introducer 200 comprises one or more hemostasis valves associated with a channel/lumen port 222. Such hemostasis valve(s) may comprise silicone or other flexible material configured to keep blood from flowing out of the channel/lumen port 222. The port 222 may serve as a tissue anchor delivery device lumen insertion port, wherein an inserted delivery device shaft may pass through the lumen 220 of the introducer 200 and out the distal end 221 thereof for access to the target chamber. The port 222 may further be dimensioned to accommodate insertion of the dilator device 228 used to guide the introducer into the target chamber (e.g., left ventricle, off-apex). The distal end 221 of the introducer 200 may have a tapered shape to seal against the delivery system lumen.

The shaft 110 may present a relatively low-profile delivery device, which may be dimensioned to fit within the lumen 220 of the introducer 200. For example, the shaft 110 may have a diameter of approximately 10 mm (about 0.5 inch) or less. Furthermore, the tip (e.g., end effector) 114 may advantageously be flexible to allow for insertion into the lumen 220 even where the lumen 220 has a smaller diameter than the extended diameter of the tip 114.

Once a suitable entry point has been established, the surgeon can use one or more sutures to make a series of stitches in one or more concentric circles in the myocardium at the desired location to create a “purse-string” closure. The Seldinger technique can be used to access the left ventricle in the area surrounded by the purse-string suture by puncturing the myocardium with a small sharp hollow needle (a “trocar”) with a guidewire in the lumen of the trocar. Once the ventricle has been accessed, the guidewire can be advanced, and the trocar removed. The valved introducer 200 (e.g., the introducer 200 with dilator 228 extending through the lumen 220 of the introducer) can be advanced over the guidewire to gain access to the left ventricle. The guidewire (not shown) and dilator 228 can be removed while the introducer 200 maintains hemostasis, with or without a suitable delivery device inserted therein, throughout the procedure. Alternatively, the surgeon can make a small incision in the myocardium and insert the introducer 200 into the heart via the incision. Once the introducer is properly placed, the purse-string suture can be tightened to reduce bleeding around the lumen of the introducer.

A suitable tissue anchor delivery device, such as a delivery device as described in the '761 PCT Application and/or the '170 PCT Application, may be advanced into the body and through the valved introducer in a manner to access the left ventricle. The advancement of the device may be performed in conjunction with echo imaging and/or direct visualization (e.g., direct transblood visualization). For example, the delivery device may be advanced in conjunction with transesophageal echocardiogram (TEE) guidance or intracardiac echo (ICE) to facilitate and direct the movement and proper positioning of the device for contacting the appropriate apical region of the heart. Typical procedures for use of echo guidance are set forth in Suematsu.

In some implementations, after prepping and placing the subject under anesthesia, echo imaging, such as involving TEE (two-dimensional (2D) and/or three-dimensional (3D)), transthoracic echocardiography (TTE), ICE, and/or cardio-optic direct visualization (e.g., via infrared vision from the tip of a 7.5 F catheter), or other imaging modality, may be performed to assess the heart, heart valves, and/or tissue anchor delivery device component(s). For example, echo imaging can be used to guide positioning of tissue anchor(s) (e.g., suture knots) onto a target valve leaflet. However, guidance of an anchor delivery device shaft to the proper position on a mitral valve leaflet as performed under echo guidance can be difficult due, for example, to the level of resolution of the echo guidance technology, and/or equipment or software limitations. As certain mitral valve leaflet repair procedures using tissue anchors, as described herein, can be performed on a beating heart, motion of the beating heart, including the mitral valve leaflets, can also interfere with desired visualization under echo.

Although the procedures described herein are with reference to repairing a cardiac mitral valve or tricuspid valve by the implantation of one or more leaflet anchors and associated cord(s), the methods presented are readily adaptable for various types of tissue, leaflet, and annular repair procedures. The methods described herein, for example, can be performed to selectively approximate two or more portions of tissue to limit a gap between the portions. That is, in general, the methods herein are described with reference to a mitral valve but should not be understood to be limited to procedures involving the mitral valve.

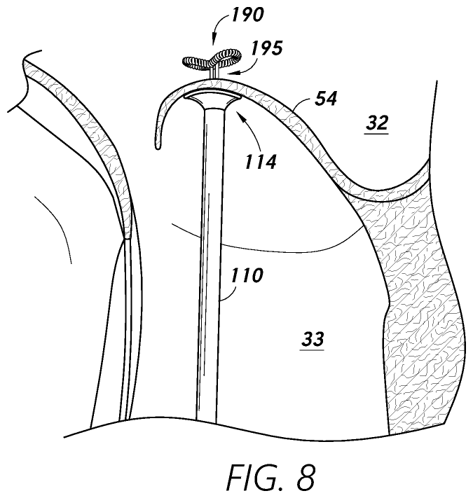

FIG. 6 shows a close-up view of a shaft 110 of a tissue anchor delivery device 100 inserted into a ventricle 33 (e.g., left ventricle) and approximated to a target valve leaflet 54 in connection with a valve-repair procedure in accordance with one or more instances of the present disclosure. For example, the valve 36 may be a mitral valve. The anchor delivery device shaft 110 can be configured to deliver a tissue anchor, such as a bulky knot to the valve leaflet 54. As an example, FIG. 6 shows a valve leaflet 54, which may represent a posterolateral leaflet of a mitral valve. It will be understood that the anchor delivery device shaft 110 can also or alternatively deliver a tissue anchor to the anteromedial mitral valve leaflet. Although the description of FIGS. 6-9 below is presented in the context of a mitral valve, it should be understood that the principles disclosed herein are applicable to other valves or biological tissues, such as a tricuspid valve.

With reference to FIGS. 3 and 6-9, the anchor delivery device shaft 110 can comprise one or more elongate lumens configured to allow delivery of one or more anchors to the valve leaflet 54. The shaft 110 can be configured to facilitate performance of one or more functions, such as grasping, suctioning, irrigating, cutting, suturing, or otherwise engaging a valve leaflet. The distal end, or tip, 114 of the shaft 110 can be configured to contact the mitral valve leaflet 54 without substantially damaging the leaflet to facilitate repair of the valve 36. For example, during a valve-repair procedure, a handle (e.g., handle 120) coupled to the shaft 110 can be manipulated in such a manner so that the leaflet 54 is contacted with the functional distal portion of the shaft 110 and a repair effectuated.

Echo imaging guidance, such as transesophageal echocardiogram (TEE) (2D and/or 3D), transthoracic echocardiogram (TTE), and/or intracardiac echo (ICE), may be used to assist in the advancement and desired positioning of the anchor delivery device shaft 110 within the ventricle 33. The distal end 114 of the shaft 110 can contact a proximal surface (e.g., underside surface with respect to the illustrated orientation of FIGS. 3 and 6-9) of the mitral valve leaflet 54, without or substantially without damaging the leaflet 54. For example, the end/tip portion or component 114 can have a relatively blunt form or configuration. The end/tip portion or component 114 can be configured to maintain contact with the proximal side of the valve leaflet 54 as the heart beats to facilitate reliable delivery of the anchor 191/190 to the target site on the leaflet 54.

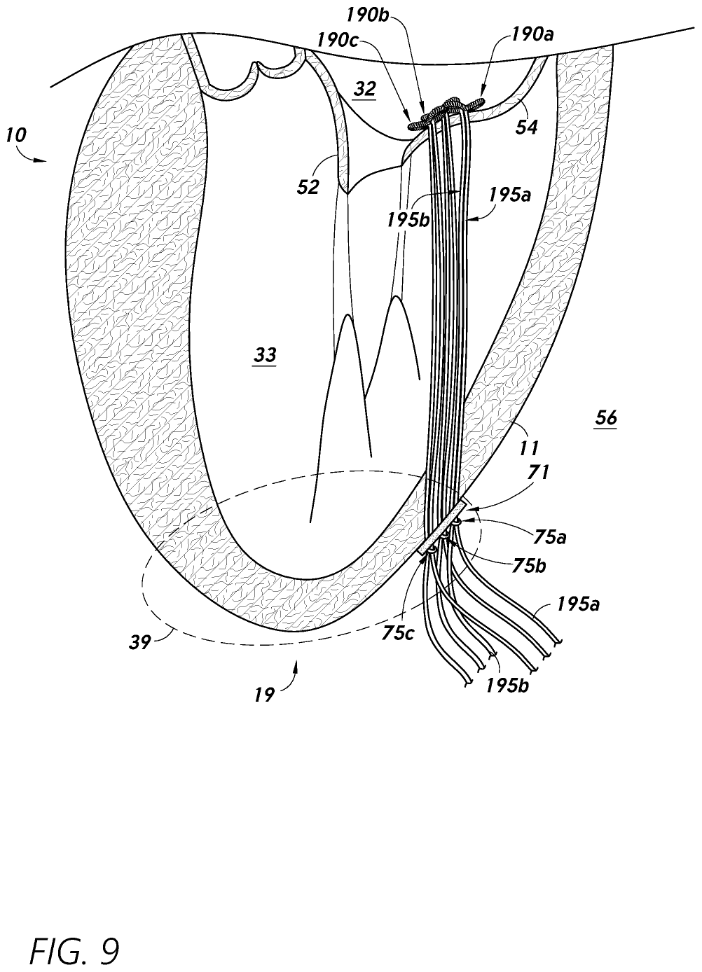

In some instances, one or more perforation devices 130 (e.g., needle(s)) can be delivered through one or more working lumens (not shown) of the shaft 110 to the valve leaflet 54 to puncture the valve leaflet 54 and project one or more sutureforms 191 including a plurality of winds of suture about a distal portion of a needle 130 into the atrium 32 (see FIG. 7), wherein the one or more sutureforms are deployed to form one or more bulky knot tissue anchors 190 (shown, e.g., in FIGS. 8 and 9). For example, as shown in FIG. 7, one or more slotted needles 130 can be deployed from the distal end of the shaft 110, thereby puncturing the leaflet 54 and projecting into the atrium 32, wherein each of the one or more slotted needles 130 is configured to be wrapped with a sutureform 191 (e.g., PTFE suture) in a particular configuration (see the '761 PCT Application for further detail regarding example suture wrapping configurations and needles for use in suture anchor deployment devices and methods). In some instances, one or more pushers and/or hollow guide wires (not shown) may be provided on or at least partially around the one or more needles 130 within the shaft 110, such that the one or more needles may be withdrawn, leaving the pushers and wound sutureforms 191. When a withdrawal force is applied to the one or more sutureforms 191 using the one or more pushers, the sutureforms 191 may form one or more bulky-knot-type anchors (e.g., the anchor 190), after which the pushers may be withdrawn, leaving the permanent knots 190 to anchor the suture(s) 195 to the leaflet 54.

FIG. 6 shows the shaft 110 of the tissue anchor delivery device 100 positioned on the target valve leaflet 54 (e.g., mitral valve leaflet). For example, the target site of the valve 54 may be slowly approached from the ventricle side thereof by advancing the distal end of the shaft 110 along or near to the posterior wall of the ventricle 33 (e.g., left ventricle) without contacting the ventricle wall. Successful targeting and contacting of the target location on the leaflet 54 can depend at least in part on accurate visualization of the shaft 110 and/or tip/end effector 114 throughout the process of advancing the tip 114 to the target site. Generally, echocardiographic equipment may be used to provide the necessary or desired intra-operative visualization of the shaft 110 and/or tip 114.

Once the tip 114 is positioned in the desired position, the distal end of the shaft 110 and the tip 114 may be used to drape, or “tent,” the leaflet 54 to better secure the tip 114 in the desired position, as shown in FIG. 6. Draping/tenting may advantageously facilitate contact of the tip 114 with the leaflet 54 throughout one or more cardiac cycles, to thereby provide more secure or proper deployment of leaflet anchor(s). The target location may advantageously be located relatively close to the free edge of the target leaflet 54 to minimize the likelihood of undesirable intra-atrial wall deployment of the anchor. Navigation of the tip 114 to the desired location on the underside of the target valve leaflet 54 may be assisted using echo imaging, as described in detail herein. Echo imaging may be relied upon to confirm correct positioning of the tip 114 prior to anchor/knot deployment.

With the shaft 110 positioned against the target leaflet 54, the plunger of the tissue anchor delivery device 100 can be actuated to move the one or more needles 130 and/or one or more pushers disposed within the shaft 110, such that the coiled sutureform portions 191 of the suture anchors slide off the needles 130. As the plunger is actuated, distal piercing portions of the needles 130 puncture the leaflet 54 and form one or more openings in the leaflet. FIG. 7 shows a close-up view of the distal portion of the delivery device shaft 110 showing a needle 130 and tissue anchor sutureform 191 projected therefrom through the target leaflet 54 in accordance with one or more instances. In some instances, a needle 130 may be configured to be projected a distance of between about 5-8 mm (about 0.2-0.3 inch), or less, distally beyond the distal end of the shaft 110 (e.g., beyond the tip 114). In some instances, a needle 130 may be projected a distance of between about 4-10 mm (about 0.15-0.4 inch). In some instances, a needle 130 may be projected a distance of about 25 mm (about 1 inch), or greater. In some instances, a needle 130 may be configured to extend until a distal tip of the needle (e.g., a pointed tip) and/or the entire coiled sutureform 191 extend through the leaflet 54. The needle 130 and/or the distal tip of the needle may be configured to pierce and/or penetrate a leaflet of a heart valve. While one or more needles 130 and/or sutureforms 191 may be projected into the atrial side 32 of the leaflet 54, the shaft 110 and/or tip 114 may be advantageously configured to remain entirely on the ventricular side 33 of the leaflet 54. In some instances, one or more needles 130 may be configured to extend a greater distance from the shaft 110 than one or more other needles. For example, a first needle may be longer than a second needle. In another example, a first needle may be situated further along in the shaft 110 than a second needle. In another example, a first needle may be deployed before a second needle. In these examples, the second needle may be situated at least partially within the shaft 110 while the first needle pierces and/or passes through the leaflet 54.

As one or more pushers (not shown) within the tissue anchor delivery device shaft 110 are moved distally, distal ends of the pushers may advantageously move and/or push the distal coiled sutureforms 191 (e.g., pre-deployment coiled portions of the suture anchors) over the distal ends of the needles 130 and further within the atrium 32 of the heart on a distal side of the leaflet 54, such that the sutureforms extend distally beyond distal ends of the needles 130. For example, in some instances, at least half a length of a sutureform 191 may be configured to extend beyond the distal end of a needle 130. The pushers may be configured to press against the sutureforms 191 and/or the needles 130 may be configured to press the sutureforms 191 against the pushers. In some instances, at least three quarters of the length of a sutureform 191 may extend beyond the distal end of a needle 130. In some instances, an entire coiled sutureform 191 may be configured to extend beyond a distal end of a needle 130.

FIG. 8 provides a close-up view of the formed suture anchor 190 on the atrial side 32 of the leaflet 54. As shown in FIG. 8, after a sutureform 191 has been pushed off and/or removed from a needle 130, pulling one or more of the suture tail(s) 195 (e.g., suture strands extending from the coiled portion of the suture) associated with the tissue anchor 190 proximally can cause the sutureform 191 to form a bulky knot anchor 190. For example, the bulky knot suture anchor 190 may be formed by approximating opposite ends of the coils of the sutureform 191 (see FIG. 7) towards each other to form one or more loops. After the sutureform 191 has been formed into the bulky knot 190, the delivery device 100 can be withdrawn proximally, leaving the tissue anchor 190 disposed on the distal atrial side of the leaflet 54, as shown in FIG. 9. In some instances, two suture tails 195 for each bulky knot 190 may extend from the proximal/ventricle side 33 of the leaflet 54 and out of the heart 1. For example, the delivery device shaft 110 can be slid/withdrawn over the suture tail(s) 195.

FIG. 9 shows a cutaway view of multiple deployed leaflet anchors 190a, 190b, 190c in accordance with one or more instances of the present disclosure. While FIG. 9 shows three deployed leaflet anchors 190, any number of leaflet anchors may be deployed. A first leaflet anchor 190a, second leaflet anchor 190b, and/or third leaflet anchor 190c may be delivered via the delivery shaft 110 simultaneously or sequentially. In some instances, the leaflet anchors 190 may each be delivered via the same delivery shaft 110 without requiring removal of the delivery shaft 110. In other words, the delivery shaft 110 may be configured to delivered into the ventricle 33 and/or into contact with the leaflet 54 and deploy multiple leaflet anchors 190 before removal of the delivery shaft 110 from the ventricle 33 and/or leaflet 54. In some instances, each of the leaflet anchors 190 may be configured to be delivered via a separate lumen of the delivery shaft 110 and/or a separate needle 130. Moreover, each of the leaflet anchors 190 may be delivered through separate holes created in the leaflet 54.

The suture tails 195 coupled to the anchors 190 may be secured at the desired tension using a pledget 71 or other suture-fixing/locking device or mechanism on the outside of the heart through which the suture tails 195 may run. For example, a first suture tail 195a and/or a second suture tail 195b of a first leaflet anchor 190a may form a first knot 75a to secure the first leaflet anchor 190a to the pledget 71. While multiple leaflet anchors 190 may be secured using a single pledget 71 as shown in FIG. 9, multiple pledgets 71 may alternatively be used. For example, each knot 75 may secure to a separate pledget 71. The knot 75 and/or other suture fixation mechanism or device may be implemented to hold the sutures at the desired tension and to the pledget 71. With the suture tail(s) 195 fixed to the ventricle wall 18, a ventricular portion of the suture tail(s) 195 (e.g., the portion of the suture tail 195 that is within the ventricle 33) may advantageously function as replacement leaflet cords (e.g., chordae tendineae) that may be configured to tether the target leaflet 54 in a desired manner. As shown in FIG. 9, the pledget 71 and/or one or more knots 75 may be configured to be situated at least partially external to the ventricle 33 and/or within an outer chamber 56.

In certain instances, the pledget 71 may be a low-porosity and/or relatively stiff pledget. Such a pledget may advantageously allow for the desired tension of the suture tails 195a to be sustained over an extended post-operative period of time. In some instances, suture tying and/or fixation may be implemented using one or more soft tissue retractors and/or right-angle clamps, which may have rubber shods associated therewith to reduce the risk of damage to the replacement cords.

In certain implementations, testing of location and/or tension of the anchor 190 and/or suture tail(s) 195 may be performed by gently tensioning the suture tails until leaflet motion is felt and/or observed. Echo imaging technology may be used to view and verify the anchor placement and resulting leaflet function. The steps and processes outlined above for placing one or more suture-knot-type tissue anchors may be repeated as necessary until the desired number of anchors have been implanted on the target valve leaflet. In some implementations, tension adjustment in the suture tail(s)/cord(s) associated with multiple leaflet anchors may be performed simultaneously. The appropriate number of leaflet anchors may advantageously be determined to produce the desired coaptation of the target valve leaflets 54, 52. All deployed leaflet anchors may advantageously be below the surface of coaptation. With respect to posterior mitral valve leaflet repair, the anterior leaflet may advantageously touch the posterior leaflet basal to the leaflet anchor(s). The pledget 71 may be drawn against the epicardial surface, and all the suture tails/cords 195 may be inserted through a tourniquet so that all cords can be tension to the desired effective coaptation together.

In some implementations, one or more leaflet anchors may be deployed in each of the mitral valve leaflets, and/or sutures/cords coupled to separate leaflets may be secured together in the heart by tying them together with knots and/or by another suitable attachment device, creating an edge-to-edge repair to decrease the septal-lateral distance of the mitral valve orifice.

With further reference to FIGS. 2-9, generally, the shaft 110 of the tissue anchor delivery device 100 may be slowly advanced into the introducer 200 until the tip 114 has flushed the introducer 200 and entered the ventricle 33. In so doing, it may be desirable to advance the shaft 110 within the ventricle 33 in such a way as to avoid traversing areas populated by papillary muscles and/or associated chordae tendineae to avoid entanglement therewith. In order to facilitate or ensure avoidance of such anatomy, imaging technology may advantageously be implemented to provide at least partial visibility of the shaft 110 within the ventricle 33, as well as certain anatomical features within the ventricle. With respect to visibility of the shaft 110 in the ventricle 33, echogenic characteristics of the shaft 110 can affect the visibility thereof using echo imaging modalities. Therefore, a shaft 110 having relatively high echogenicity as described in detail herein may advantageously allow for more accurate and/or simplified advancement of the shaft 110, as well as placement of the tip 114 at the target implantation site at the valve leaflet 54 (e.g., an anterior or posterior leaflet of a mitral valve). In some implementations, hybrid imaging technologies may be used, wherein echo imaging is used in combination with a different imaging modality. Multi-imaging modalities may provide improved visibility of anatomical and/or delivery system components.

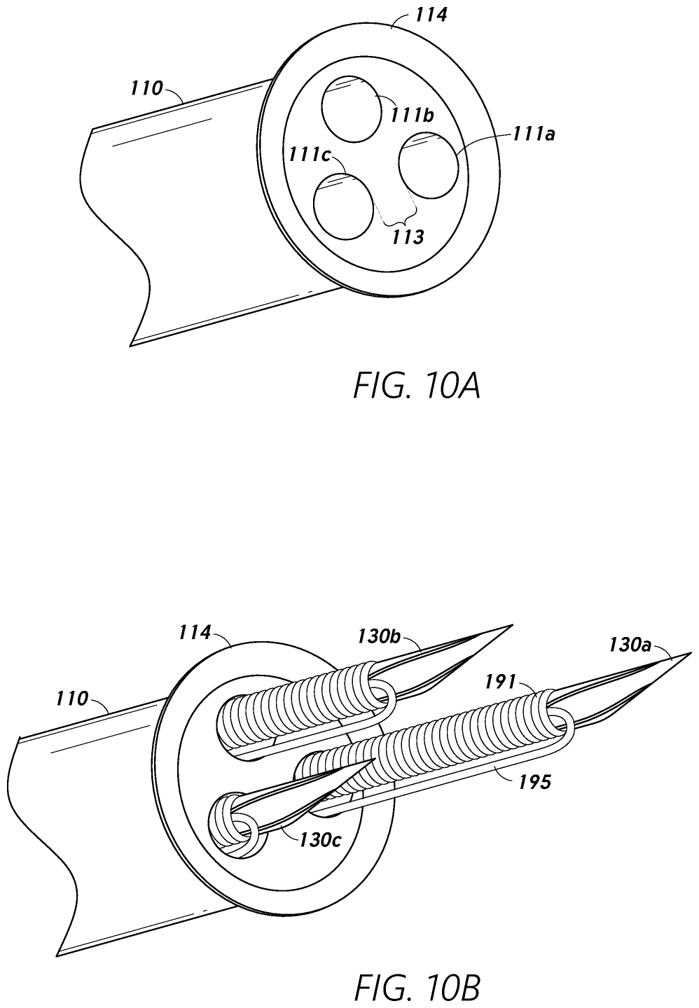

FIG. 10A provides a perspective view of a distal portion of a multi-lumen medical device shaft 110 in accordance with one or more examples. The illustrated multi-lumen shaft 110 may be configured to deliver and/or slidably receive more than one needle 130, which allows for the placement of multiple repair devices (e.g., sutureforms 191), thus reducing the number of penetrations needed through the valve introducer. The shaft 110 may comprise a rigid elongate tube forming multiple (e.g., three) internal lumens 111, as shown. FIG. 10A illustrates an instance of a shaft 110 comprising three lumens 111a, 111b, 111c. However, the shaft 110 may alternatively comprise two lumens 111 or more than three lumens 111. The lumens 111 may have circular shapes, as illustrated, or may have any other shape. Each of the lumens 111 may be configured to slidably receive one needle 130. In some instances, the lumens 111 are substantially equal in size or volume, whereas in other instances, one of the lumens (e.g., a first lumen 111a) may be greater in size or volume than the other.

The multiple lumens 111 may be configured to prevent contact and/or create separation between needles and/or anchors within the lumens 111. For example, the lumens in may extend an entire interior length of the shaft 110 such that as the needles 130 slide through the shaft 110, the needles 130 are not in contact with each other. When the needles 130 exit the distal end of the shaft 110, the needles 130 may be sufficiently separated due to the position of the lumens 111 that the needles 130 contact and/or pierce the valve leaflet at different points.

Moreover, in some instances the shaft 110 may have any size and/or shape. For example, the shaft 110 may have the generally cylindrical/circular shape shown in FIGS. 10A and 10B. However, the shaft 110 may alternatively have an ovular, rectangular, or other shape. In some instances, the size and/or shape of the lumens 111 may correspond to the size and/or shape of the shaft 110. For example, one or more lumens 111 may have a generally cylindrical/circular shape to conform to the rounded sides of the shaft 110. In some instances, the shaft 110 may have a diameter of approximately ¼ inch (about 6 mm) or less. In some instances, one or more lumens 111 may have a size and/or shape configured to fit a needle carrying a sutureform.

In some examples, the shaft 110 may comprise a tip portion 114 configured to contact a leaflet of a heart without and/or substantially without damaging the leaflet. The tip portion 114 may extend along the entire outer surface of the distal end of the shaft 110 such that only the tip portion 114 of the shaft 110 may contact the leaflet.

Each lumen 111 may pass an entire length of the shaft 110 or may extend only partially along the length of the chamber. In some instances, each lumen 111 may have a corresponding opening at a proximal portion of the shaft 110 (e.g., where the shaft 110 meets a handle portion) as well as at the distal portion (shown in FIG. 10A). In optional examples, each lumen may be a branch lumen from a common base lumen.

In some instances, the one or more lumens 111 may be oriented around a central point of the chamber. For example, each of the one or more lumens 111 may be an equal distance from a center point of the distal portion of the shaft 110. Moreover, each lumen may be separated from other lumens by a desired distance to create sufficient separation between the lumens 111 to prevent multiple needles from entering a common hole in the leaflet. In some instances, a first lumen 111a may be separated from a second lumen 111b and/or a third lumen 111c by a first distance 113. Similarly, the second lumen 111b may be separated from the third lumen 111c by the first distance 113. In some examples, the first distance 113 may be at least ¼ mm and/or up to 2 or 3 mm. The first distance 113 may be selected as desired as a tradeoff between desirably creating separation between the needles while limiting the profile and/or size of the shaft 110 to minimize invasiveness of insertion of the shaft 110. Accordingly, the first distance 113 may be maximized while maintaining an acceptable diameter (e.g., about ¼ inch (about 6 mm) or less) of the shaft 110.

FIG. 10B is a perspective view of a distal portion of a multi-lumen medical device shaft 110 having multiple needles 130 and sutureforms 191 projected from the distal portion of the shaft 110 in accordance with one or more instances. Each interior lumen 111 of the multi-lumen shaft 110 may be configured to hold one of the multiple needles 130. Each of the sutureforms 191 may have a coiled configuration about one of the needles 130, which may advantageously be slotted needles.

In some instances, one or more of the needles 130 may be composed at least partially of a shape-memory alloy (e.g., nitinol) and/or may otherwise be shaped such that the needles 130 curve away from each other after exiting the shaft 110. For example, at least a distal portion of the first needle 130a may be at least partially composed of nitinol and/or may be shape-set to curve and/or otherwise create greater distance between the first needle 130a and the second needle 130b and/or the third needle 130c. The second needle 130b and/or third needle 130c may be similarly shape-set to curve away from the first needle 130a and/or each other. In this way, the risk of the needles 130 entering a puncture created by a different needle 130 may be reduced and/or the delivered leaflet anchors may have greater separation to spread out the tension force applied by the artificial cords attached to the anchors.

As shown, the sutureform 191 may comprise a plurality of winds of a suture around at least a portion of a needle 130. Each lumen 111 may further be used to hold at least a portion of a pusher device (not shown; described above), which may be used to deploy the needle 130 and/or deploy the sutureform 191 from the needle 130.

By including multiple needles 130 in the shaft 110, only a single shaft 110 may be required to be inserted into the body to deliver multiple needles 130 and/or multiple sutureforms 191 to a valve leaflet. Once the shaft 110 is in place and/or the tip portion 114 is in contact with the valve leaflet, the one or more needles 130 may be extended through the one or more lumens 111 and/or beyond the distal portion of the shaft 110. As shown in FIG. 10B, a first needle 130a may be extended out of the terminal end of the chamber a greater distance than a second needle 130b and/or a third needle 130c. Each of the needles 130 may be extended through activation of a common plunger (not shown; see FIGS. 2 and 3) or similar device and/or each needle 130 may be individually extended using a different plunger or similar device. In examples in which all of the one or more needles 130 are extended using a single plunger, the first needle 130a may be longer than the second needle 130b and/or the second needle 130b may be longer than the third needle 130c. Additionally or alternatively, the first needle 130a may be positioned within the first lumen 111a further along in the shaft 110 than the second needle 130b. For example, the first needle 130a may be pre-extended a small distance such that when a plunger or similar device is activated, the first needle 130a may contact a valve leaflet before the second needle 130b and/or third needle 130c contacts the valve leaflet.

In some examples, the shaft may be configured to be twisted to rotate positions of the needles within the shaft. For example, after the first needle 130a penetrates the valve leaflet, the shaft 110 may be rotated as desired to position the second needle 130b and/or third needle 130c below an edge portion of the valve leaflet and/or other desired position of the valve leaflet.

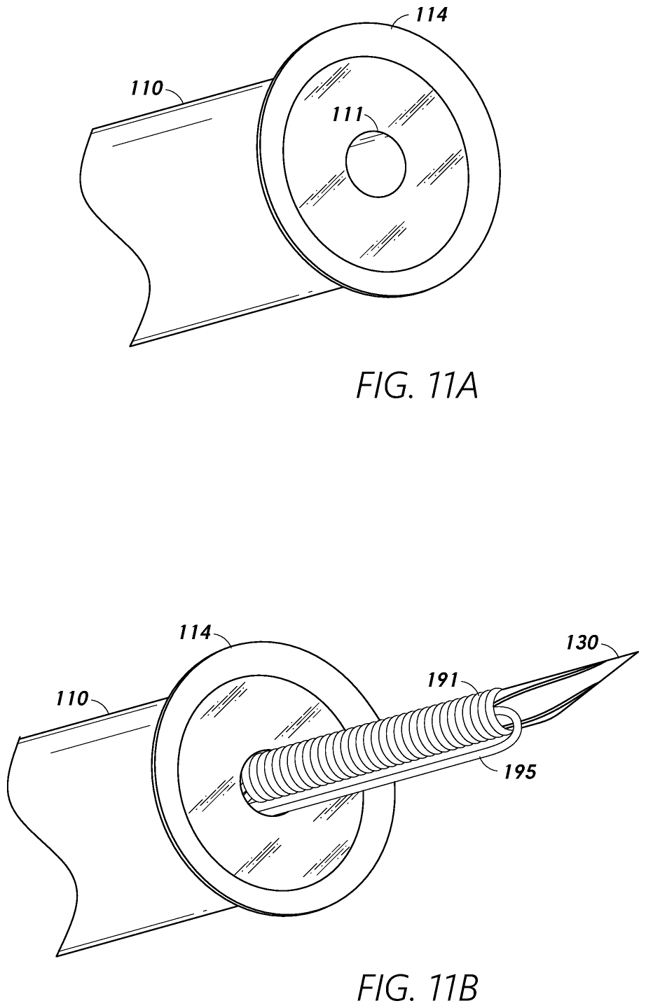

FIG. 11A provides a perspective view of a distal portion of another medical device shaft 110 configured for delivery of multiple delivery devices (e.g., needles) and/or implants (e.g., suture anchors) in accordance with one or more examples. The illustrated shaft 110 may be configured to deliver and/or slidably receive more than one needle 130, which can enable the placement of multiple repair devices (e.g., sutureforms 191), thus reducing the number of penetrations needed through the valve introducer. The shaft 110 may comprise a rigid elongate tube forming one or more internal lumens 111. While only a single opening (e.g., lumen 111) is shown at a distal end portion of the shaft 110, the shaft 110 may comprise any number of lumens 111 and/or one or more lumens 111 may converge and/or branch within the shaft 110. The shaft 110 may comprise two lumens 111, three lumens 111, or more than three lumens 111. The lumens 111 may have circular shapes, as illustrated, or may have any other shape. Each of the lumens 111 may be configured to slidably receive one or more needles 130. In some instances, the lumens 111 may be substantially equal in size or volume, whereas in other instances, some lumens 111 may be greater in size or volume than others.

In some instances, a single opening into a lumen 111 may be configured to allow a single needle 130 to exit the shaft 110 at a time and/or to prevent multiple needles 130 from exiting the shaft 110 simultaneously. For example, the opening into the lumen 111 may have a diameter sufficient for only a single needle 130 and/or leaflet anchor 191 to fit and/or pass through the opening at a time. The lumen 111 may gradually increase in diameter from a minimal diameter at or near the opening in to a maximal diameter along a midsection of the shaft 110 to accommodate multiple needles 130 and/or leaflet anchors 191 and/or decrease in diameter at or near the opening into the lumen 111 to allow only a single needle 130 of multiple needles 130 to extend through the opening at a time. The minimal diameter may be less than the maximal diameter. For example, the maximal diameter may be sufficient to fit and/or slidably receive multiple needles 130 while the minimal diameter may be approximately equal to a needle 130 carrying a sutureform 191. In some instances, the lumen 111 may extend an entire interior length of the shaft 110 and/or may change shape and/or branch into distinct lumen portions. For example, the shaft 110 may comprise three lumen branches, each configured to receive a needle 130. The three lumen branches may merge into a single lumen 111 near the distal end portion of the shaft 110 (e.g., near the tip portion 114) to allow only a single needle 130 to exit the shaft 110 at any single point in time.

Moreover, in some instances the shaft 110 may have any size and/or shape. For example, the shaft 110 may have the generally cylindrical/circular shape shown in FIGS. 10A and 10B. However, the shaft 110 may alternatively have an ovular, rectangular, or other shape. In some instances, the size and/or shape of the lumen 111 may correspond to the size and/or shape of the shaft 110. For example, one or more lumens 111 may have a generally cylindrical/circular shape to conform to the rounded sides of the shaft 110. In some instances, the shaft 110 may have a diameter of approximately ¼ inch (about 6 mm) or less. In some instances, one or more lumens 111 may have a size and/or shape configured to fit a needle carrying a sutureform.

In some examples, the shaft 110 may comprise a tip portion 114 configured to contact a leaflet of a heart without and/or substantially without damaging the leaflet. The tip portion 114 may extend along the entire outer surface of the distal end of the shaft 110 such that only the tip portion 114 of the shaft 110 may contact the leaflet.

Each lumen 111 may pass an entire length of the shaft 110 or may extend only partially along the length of the chamber. In some instances, each lumen 111 may have a corresponding opening at a proximal portion of the shaft 110 (e.g., where the shaft 110 meets a handle portion) and/or at the distal portion. One or more lumens 111 may branch from a common base lumen. In some instances, a lumen 111 and/or opening to a lumen 111 may be situated at or near a central point of the shaft 110, as shown in FIGS. 11A and 11B.

FIG. 11B is a perspective view of a distal portion of a single-lumen medical device shaft 110 having a needle 130 and sutureform 191 projected from the distal portion of the shaft 110 in accordance with one or more instances. While only a single needle 130 is shown exiting the shaft 110, the shaft 110 may be configured to simultaneously carry multiple needles 130 and/or multiple sutureforms 191 while allowing a single needle 130 and/or a single sutureform 191 to exit a lumen 111 of the shaft 110. The interior lumen 111 of the shaft 110 may be configured to hold one or more of the multiple needles 130. Each of the sutureforms 191 may have a coiled configuration about one of the needles 130, which may advantageously be slotted needles.

As shown, the sutureform 191 may comprise a plurality of winds of a suture around at least a portion of a needle 130. The interior lumen 111 may further be used to hold at least a portion of a pusher device (not shown; described above), which may be used to deploy the needle 130 and/or deploy the sutureform 191 from the needle 130.

By delivering multiple needles 130 through a single lumen 111 of the shaft 110, only a single shaft 110 may be required to be inserted into the body to deliver multiple needles 130 and/or multiple sutureforms 191 to a valve leaflet. Once the shaft 110 is in place and/or the tip portion 114 is in contact with the valve leaflet, a first needle 130 may be extended through the lumen 111 and/or beyond the distal portion of the shaft 110. A sutureform 191 at the first needle 130 may be formed into a suture anchor at a distal side of the leaflet. The first needle 130 and/or shaft 110 may be retracted and/or repositioned along the leaflet to allow a second and/or third needle 130 to subsequently be passed through the leaflet to deliver additional suture anchors.

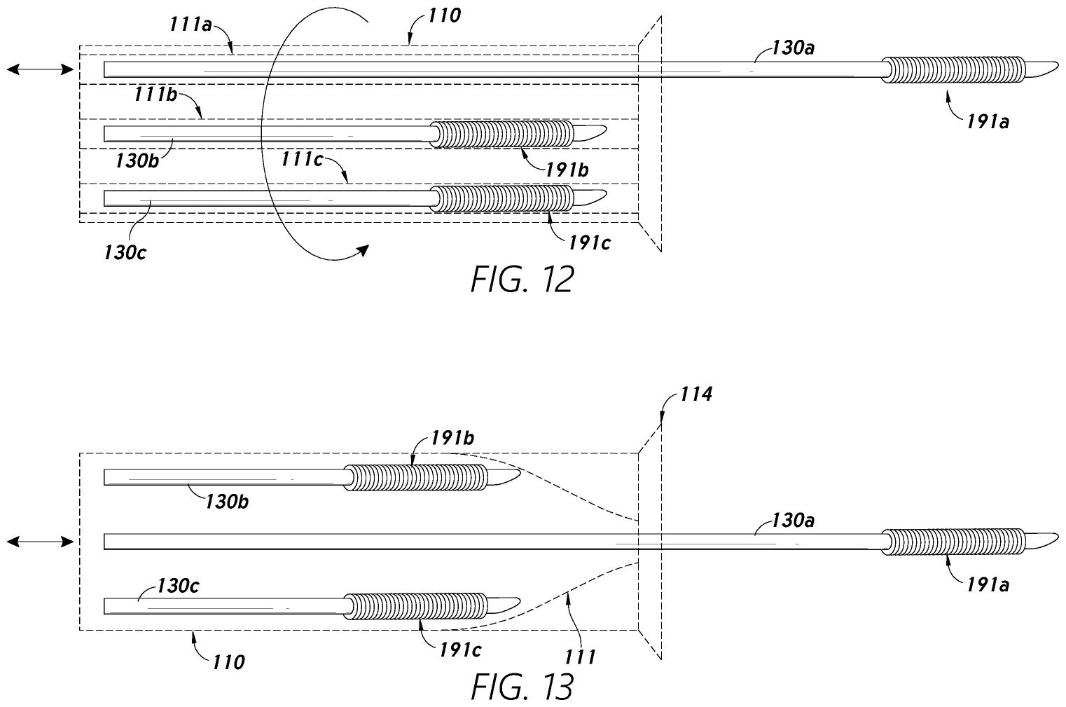

FIG. 12 illustrates a multi-lumen shaft 110 for delivering multiple needles to a valve leaflet. In some instances, a first needle 130a may be configured to extend from a first lumen 111a to contact and/or pass through the valve leaflet to deliver a first sutureform 191a to a distal side of the valve leaflet. A second needle 130b may be situated within a second lumen 111b and/or a third needle 130c may be situated within a third lumen 111c for delivery through the valve leaflet subsequent to delivery of the first needle 130a through the valve leaflet. The second needle 130b may be configured to carry a second sutureform 191b and/or the third needle 130c may be configured to carry a third sutureform 191c for placement at the valve leaflet subsequent to placement of the first sutureform 191a.

In some instances, the multi-lumen shaft 110 may be configured to be at least partially rotated to facilitate anchoring at or near a leaflet edge. In some examples, the first needle 130a delivered via the shaft 110 may be extended to pierce the leaflet at a first point. The first needle 130a may be longer than the second needle 130b and/or third needle 130c and/or the first needle 130a may be extended further than the second needle and/or third needle 130c such that the first needle 130a pierces the leaflet before the second needle 130b and third needle 130c pierce the leaflet.

After the first needle 130a pierces the leaflet, the shaft 110 may be twisted and/or rotated until the second needle 130b and/or third needle 130c is below a desired position of the leaflet that is nearer the leaflet edge than the first point. In some examples, the second needle 130b and/or third needle 130c may be configured to pierce the leaflet at approximately ½ mm to 3 mm from the leaflet edge. The first point may be a greater distance from the leaflet edge than 0.5 mm to 3 mm, and may be approximately 5 mm or 6 mm or more from the leaflet edge.

By piercing the leaflet first with the first needle 130a, the first needle 130a may be configured to maintain contact between the delivery systems and the leaflet to more easily anchor other needles at or near the leaflet edge. Because the first needle 130a can penetrate the leaflet at a greater distance from the leaflet edge than the second needle 130b and/or third needle 130c, the first needle 130a may be less likely to slip out of contact with the leaflet after delivery of the first needle 130a through the leaflet.

FIG. 13 illustrates a single-lumen shaft 110 for delivering multiple needles 130 and/or multiple leaflet anchors 191 to a valve leaflet. In some instances, a first needle 130a, second needle 130b, and/or third needle 130c may be situated within a common lumen 111 of the shaft 110. Accordingly, the lumen 111 and/or shaft 110 may be sized to fit and/or slidingly/slidably receive multiple needles 130 and/or multiple leaflet anchors 191. In some instances, the lumen 111 may have a non-constant and/or variable width and/or diameter. For example, the lumen 111 may gradually reduce in width and/or diameter at or near a distal end portion (e.g., at and/or near the atraumatic tip 114) such that the lumen 111 may allow only a single needle 130 (e.g., a first needle 130a) and/or a single leaflet anchor 191 (e.g., a first leaflet anchor 191a) to exit the shaft 110 at any one point in time.

In some instances, a first needle 130a may be configured to extend through the lumen 111 and/or out of an opening into the lumen 111 at the distal portion of the shaft 110 (e.g., at or near the atraumatic tip 114) to contact and/or pass through the valve leaflet to deliver a first sutureform 191a (e.g., a leaflet anchor) a to a distal side of the valve leaflet. A second needle 130b and/or a third needle 130c may be situated within the lumen 111 for delivery through the valve leaflet subsequent to delivery of the first needle 130a through the valve leaflet. The second needle 130b may be configured to carry and/or deliver a second sutureform 191b and/or the third needle 130c may be configured to carry and/or deliver a third sutureform 191c for placement at the valve leaflet subsequent to placement of the first sutureform 191a.

In some instances, after the first sutureform iia is delivered, the first needle 130a may be configured to be retracted into the shaft 110 and/or lumen 111 to allow other needles 130 to be passed through the distal end portion of the shaft 110 and/or lumen. In some examples, the first needle 130a delivered via the shaft 110 may be extended to pierce the leaflet at a first point.

After the first needle 130a pierces the leaflet, the shaft 110 may be moved until the at least a central portion of the shaft 110 is below a desired position of the leaflet (e.g., a portion that is nearer the leaflet edge than the first point). The second needle 130b may then be extended beyond the distal end portion of the lumen 111 and/or shaft 110 to allow the second needle 130b to pierce the leaflet. Following delivery of the second sutureform 191b, the second needle 130b may be retracted and/or the shaft may again be moved to a target point for the third needle 130c. The third needle 130c may then be extended to deliver the third sutureform 191c at the leaflet.

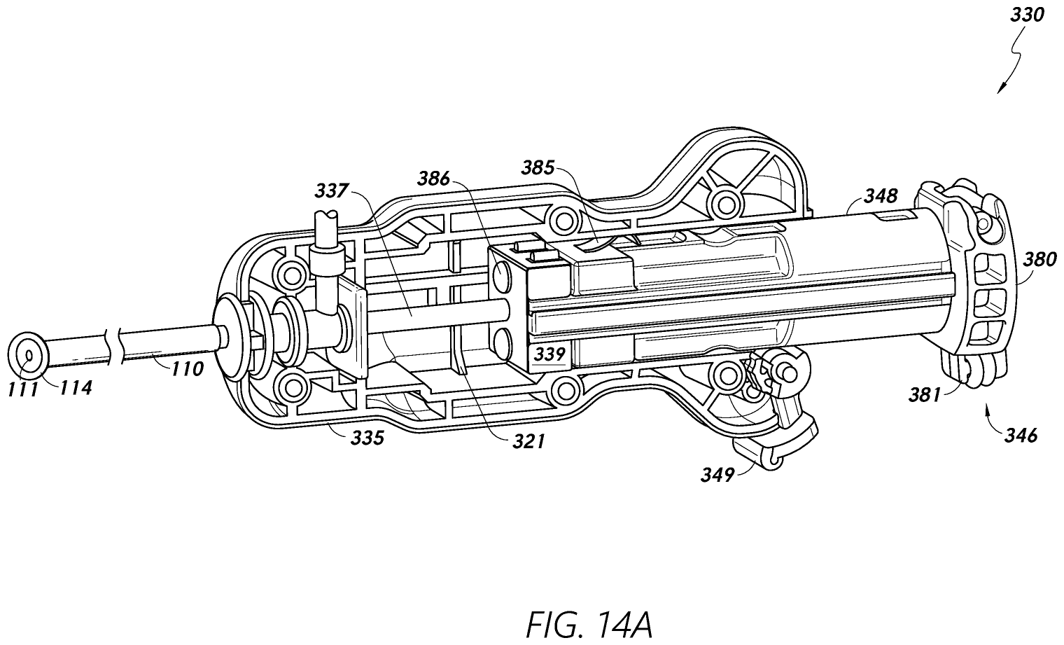

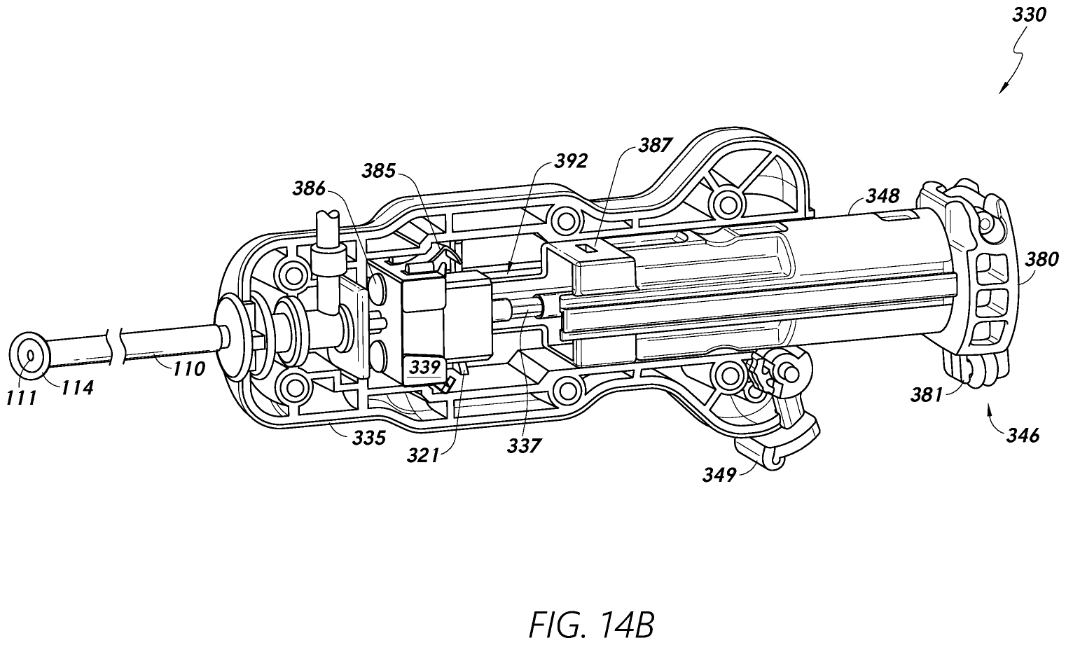

FIGS. 14A and 14B illustrate interior views of a delivery device 330 that can be used to deliver and/or form one or more leaflet anchors to be disposed on a distal side of a mitral valve leaflet in accordance with some examples. In some instances, the delivery device 330 includes an outer tube 110 (e.g., shaft) having an end effector 114 (e.g., tip portion) at a distal end of the shaft 110 that can be placed in contact with a leaflet of a mitral valve as described above. The end effector 114 can be coupled to a distal end portion of an outer tube 110 and a proximal end portion of the outer tube 110 is coupled to a handle 335 at a proximal end portion of the outer tube 110. One or more elongate pushers 337 may be movably disposed within one or more interior lumens of the outer tube 110 and/or may be coupled to a pusher hub 339 that may be movably disposed within the handle 335 and releasably coupled to a plunger 348. Each pusher 337 may be configured to slidably receive one needle 130. One or more needles may be movably disposed within the lumens of the one or more pushers 337 and/or may be coupled to a needle hub (not shown) that may also be coupled to the plunger 348. The plunger 348 may be used to actuate and/or or move the needles and/or the pushers 337 during deployment of one or more distal anchors and/or may be movably disposed at least partially within the handle 335. For example, the handle 335 may define a lumen in which the plunger 348 can be moved. During operation, the one or more pushers 337 may also move within the lumen of the handle 335 as described in more detail below. The delivery device 330 may also include a locking lever 349 that can be used to prevent the plunger 348 from moving within the handle 335 during storage and prior to performing a procedure to deploy the distal anchor.

The delivery device 330 may comprise a suture catch 346 (also referred to as “tether catch”) which may be coupled to the plunger 348 at a proximal end of the delivery device 330. The suture catch 346 can be configured to releasably hold or secure one or more sutures extending through the delivery device 330 during delivery of the one or more distal anchors as described above and as described in more detail below. In some instances, the suture catch 346 can hold the one or more sutures with a friction fit or with a clamping force and can have a lock that can be released after the distal anchors have been deployed/formed into bulky knots. The suture catch 346 includes an arm 380 and contact members 381 (e.g., silicone O-rings) coupled to the arm 380. The arm 380 can be moved from a closed position in which the contact members 381 engage the suture strands within a slot in the plunger 348, to an open position thereby allowing the proximal end portions of the sutures to be released from the suture catch 346. The delivery device 330 can then be withdrawn proximally, leaving the distal anchor disposed on the distal side of the leaflet, and the two lengths of each suture extending out of the heart, as described with respect to previous instances. When in the closed position, the arm 380 and the contact members 381 pinch or otherwise secure the sutures to prevent or otherwise limit the sutures from moving relative to the device 330. When in the open position (e.g., after delivery of the distal anchors and during removal of the device 330 from the heart), the arm 380 and the contact members 381 allow movement of the sutures relative to the device 330 such that the device 330 can be separated from the sutures, as described in more detail below.

Distal end portions of the sutures can be formed into elongated coiled configurations (e.g., sutureforms) and/or may be disposed within the outer tube 110 at the distal end portion of the delivery device 330. For example, the coils of the sutures can be provided and/or shipped disposed around the needles with the proximal most coil abutting against the sutures. Two strands of each suture may extend from the distal elongated coiled portion of the suture, extend through the lumen of the needle, through a passageway of the plunger 348 and exit the plunger 348 at a proximal end portion of the plunger 348. The distal elongated coiled portions of the sutures may be formed into distal anchors (e.g., bulky knots) upon actuation of the delivery device 330. The distal anchors (e.g., bulky knots) can be in the form of one or more multi-turn coils of the sutures that can be changed from the elongated coiled configuration during delivery to a knot configuration by approximating opposite ends of the coils towards each other, to form one or more loops.

To prepare the delivery device 330 for delivering and forming distal anchors within, for example, a left atrium of the heart to repair a mitral valve, the locking lever 349 may be released from its locked or engaged position (e.g., its position during storage of the delivery device 330) in which the plunger 348 is prevented from moving (e.g., proximally and distally) within the handle 335 to its unlocked or disengaged position in which the plunger 348 can be moved within the handle.

To deliver and form the distal anchors within, for example, a left atrium of the heart to repair a mitral valve, the distal end portion of the delivery device 330 can be inserted through an apex portion of the heart and into the left ventricle until the end effector 114 contacts a proximal side of the mitral valve leaflet. With the delivery device 330 positioned against the mitral leaflet, and with a proximal end portion of the sutures (e.g., two suture strands of each suture) secured to the suture catch 346, the plunger 348 may be actuated to move the needle hub, the needles, the pushers 337 and pusher hub 339, and the coiled portions of the sutures (e.g., the distal anchors) distally until the plunger 348 contacts a stop member 321 within the handle 335, which may limit the travel of the plunger 348 in the distal direction. As the plunger 348 is actuated, a distal piercing portion of a first needle may puncture the leaflet and form an opening in the leaflet. Because the first needle may be coupled to the plunger 348 and/or may move with the plunger 348, the distance the distal end portion of the first needle may extend within the left atrium on the distal side of the leaflet may be determined by the amount of travel allowed by the plunger 348. Thus, in this instance, the delivery device 330 may be configured to advance the distal end portion of the first needle a shorter distance, for example, between about 5-8 mm (about 0.2-0.3 inch) (e.g., about 6 mm (about 0.25 inch)), or less, distally beyond the distal end of the delivery device 330 (e.g., beyond the end effector 114). In other instances, the first needle can be extended outside the delivery device a distance of about 3-10 mm (about 0.15-0.4 inch). For example, in some instances, the first needle may extend until the distal/pointed tip of the first needle and/or a first wrap or two of the coiled suture attached to the first needle extend through the leaflet. The handle 335 may then be rotated and/or moved as needed and/or one or more additional needles and/or sutures may then be extended beyond the distal end of the delivery device 330 and/or through the leaflet. The length and/or position of the first needle may ensure that the first needle passes through the leaflet before any other needles. When the plunger 348 reaches the stop member, the pushers 337 and/or pusher hub 339 may be released from the plunger 348 and/or may be advanced further distally to a distal position within the handle 335.

FIG. 14B illustrates the delivery device 330 with the pusher hub 339 separated from the plunger 348 to show the needles and pushers 337 passing through the pusher hub 339. The pusher hub 339 may be configured to complimentarily mate with an opening 392 defined by and located at a distal end portion of the plunger 348. In use, prior to deployment, a proximal end portion of the pusher hub 339 may be disposed within the opening 392 defined by the plunger 348. In this position, a pair of tabs 385 of a spring member 386 coupled to the pusher hub 339 are disposed within slots 387 defined by the plunger 348. For example, the spring member 386 can have a biased configuration in which the tabs 385 are disposed in an open position, and when the pusher hub 339 is coupled to the plunger 348 the tabs 385 are compressed by the inner walls of the handle 335, which define a first portion of a passageway within the handle 335. As the pusher hub 339 is moved distally, the spring member 386 will slide into a second portion of the passageway of the handle 335 which has a larger size, allowing the tabs 385 to move to their biased open configuration and disengaging the tabs 385 from the slots 387 of the plunger 348.

When the plunger 348 is actuated (e.g., moved distally within the handle 335), the pusher hub 339 may move distally with the plunger 348 until the plunger 348 reaches the stop member 321, at which point the pusher hub 339 is moved into the second portion of the passageway of the handle 335 as described above, allowing the tabs 385 on the spring member 386 to disengage or release the pusher hub 339 from the plunger 348. Upon release from the plunger 348, a pusher 337 and the pusher hub 339 are advanced further distally to a distal position within the handle 335, in response to a force provided by a biasing member (e.g., a compression spring) disposed within a lumen of the plunger 348. The biasing member may be coupled to and disposed between the pusher hub 339 and the needle hub. In this manner, with use of the biasing member, a desirable and repeatable force can be applied to the pusher 337 and the pusher hub 339, resulting in a desirable and repeatable delivery of the distal anchors.

Although the lumen of the handle 335 is shown in this instance as being rectangular, in some instances, the lumen of the handle can have any suitable shape (e.g., a circular or semi-circular shape). In such instances, the components that cooperatively function within the handle 335 (e.g., the pusher 337, the pusher hub 339, the plunger 348), as described above with respect the delivery device 330, can be suitably sized and/or shaped to cooperatively function with any shape and/or size selected for a particular handle and lumen defined therein.

In use, as the plunger 348 may be actuated to move the pusher 337 and the pusher hub 339 distally within the handle 335, the plunger 348 can reach the stop member 321 at which point in time a spring member may slide into the second portion of the passageway of the handle which has the larger size, allowing the tabs 385 to move to their biased open configuration and disengaging the tabs 385 from the slots 387 of the plunger 348. In this manner, a biasing member may be released from its compressed configuration and transition towards a biased uncompressed configuration thereby resulting in travel of the pusher 337 and the pusher hub 339 distally within the handle 335. As the pusher 337 are moved distally, distal ends of the pusher 337 may move and/or push the distal coiled portion of the sutures and/or distal anchors over the distal ends of the needles and further within the left atrium of the heart on a distal side of the mitral leaflet, such that the coiled portions of the sutures extend distally beyond distal ends of the needles. For example, in some instances, at least half a length of the distal coiled portions of the sutures extends beyond the distal ends of the needles. In some instances, at least three quarters of the length of the distal coiled portions of the sutures extends beyond the distal end of the needles. In other instances, the entire coiled portions of the sutures extend beyond the distal ends of the needles. To allow the distal coiled portions of the sutures and/or distal anchors to slide relative to the plunger 348, when the sutures are loaded within the delivery device 330, there may be slack in the sutures between the distal coiled portions of the sutures and the suture lock within the suture catch 346.

After the distal coiled portions of the sutures are moved to the distal side of the leaflet, the plunger 348 may be released to allow the plunger 348 to move proximally, which moves or pushes the needles and suture catch 346 proximally. For example, in some instances, the plunger 348 can be actuated by the user manually pushing the plunger distally within the handle 335 with for example a thumb or finger. To release the plunger 348, the user can release his thumb which allows the plunger 348 to be moved back proximally. For example, in some instances, when the user releases his thumb from the plunger 348, a biasing member (e.g., a spring; not shown) can push the plunger 348 back in the proximal direction. When the suture catch 346 is moved proximally, this in turn pulls the sutures (e.g., suture strands extending from the coiled portion of the suture) proximally to form the bulky knot configuration of the distal anchor against the distal end of the pusher 337. For example, as described above, the bulky knot may be formed by approximating opposite ends of the coils of the elongated coil portion of a suture towards each other, to form one or more loops. After the distal anchor has formed a knot, the proximal end portions of the sutures can be released from the suture catch 346. The delivery device 330 can then be withdrawn proximally, leaving the distal anchors disposed on the distal side of the leaflet, and two lengths or strands of the sutures extending from the proximal side of the leaflet and out of the heart. In other words, with the sutures released from the suture catch 346 the delivery device 330 can be slid over the sutures for removal.

As described above for previous instances, the lengths or strands of the sutures between the distal anchors and the opening in the heart can be adjusted until the desired length is established. The proximal ends of the sutures can then be secured to an outer surface of the heart at, for example, the apex, with one or more proximal anchors (not shown). The proximal anchor can be, for example, a pledget, one or more knots, or other suitable anchoring device. The above procedure can be performed multiple times on the same leaflet, and/or can be performed on the other mitral valve leaflet in the same manner. The result can thus be that one or more anchor-tether apparatuses (e.g., anchor-tether apparatus) as described above are each anchored on a distal side of a leaflet with distal anchors and secured to the apex of the heart with proximal anchors via the tether. Alternatively, if one or more anchor-tether apparatuses are attached to both mitral valve leaflets, an anchor-tether apparatus attached to each leaflet can be secured together in the heart by tying them together with knots or by another suitable attachment member, creating an edge-to-edge repair to decrease the septal-lateral distance of the mitral valve orifice. The two attached anchor-tether apparatuses can be left loose or tensioned to create a “facilitated” edge-to-edge repair before being secured to an outer surface of the heart with a proximal anchor.

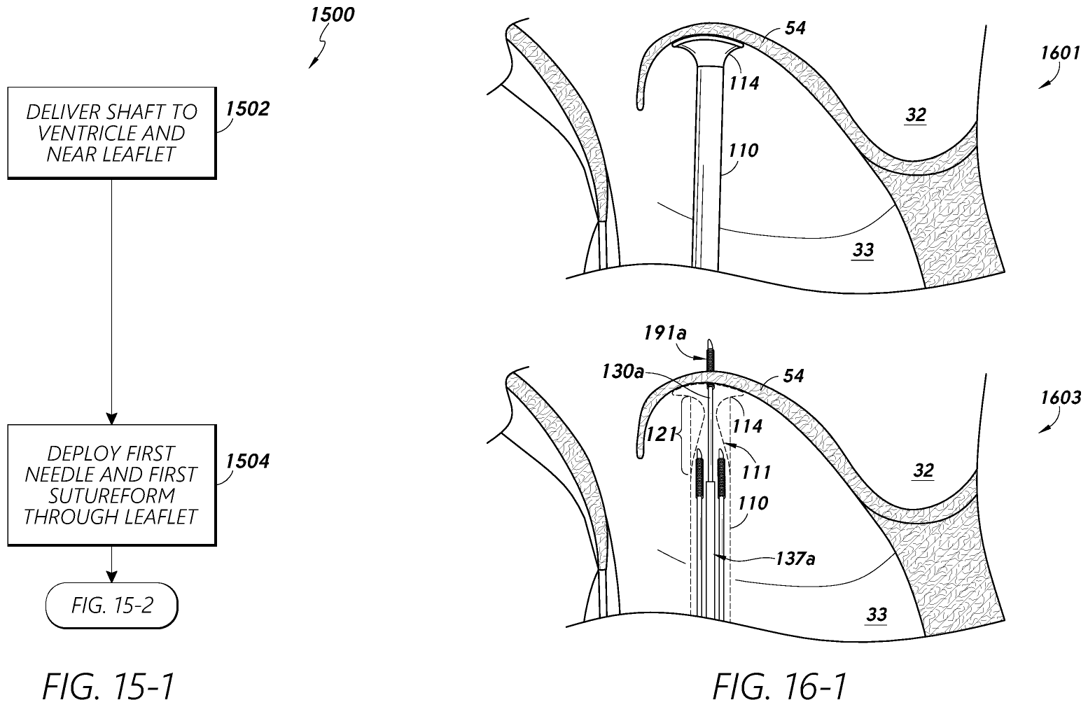

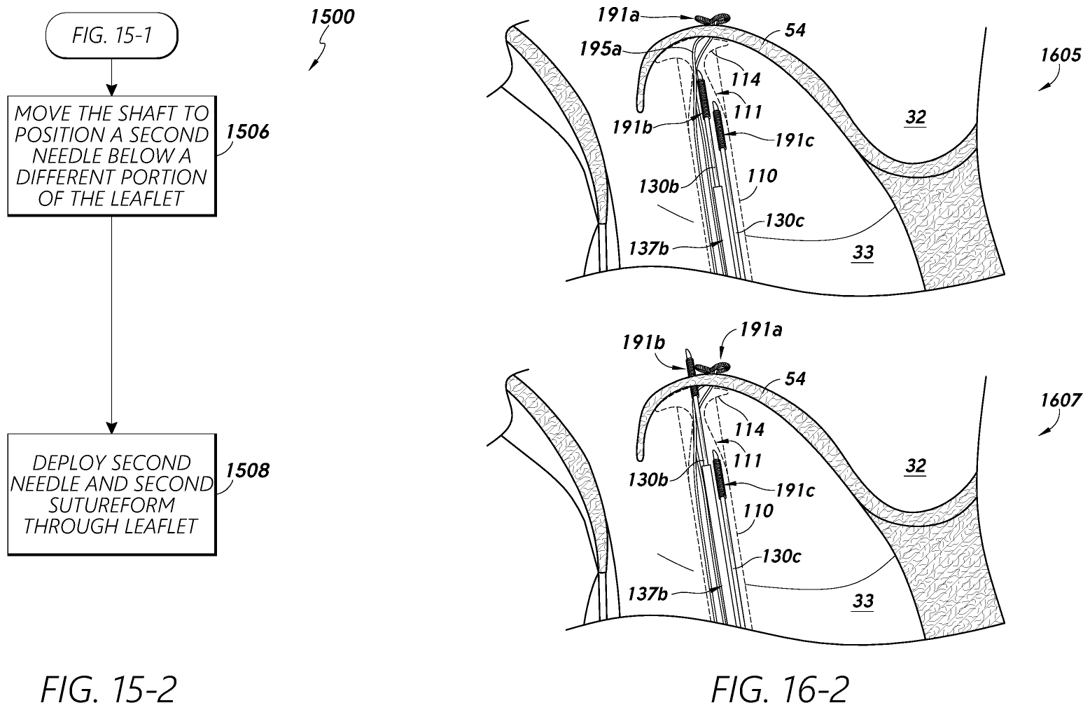

FIG. 15 (FIGS. 15-1 and 15-2) is a flow diagram illustrating a process for delivering multiple leaflet anchors to a valve leaflet in accordance with one or more instances of the present disclosure. FIG. 16 (FIGS. 16-1 and 16-2) provides illustrations corresponding to steps of the process described in FIG. 15.

At block 1502, the process 1500 involves delivering a shaft of a delivery device (see FIG. 2) through a wall of a heart (e.g., the apex region) into a ventricle 33 and into contact with a leaflet 54 of a valve of the heart, as shown in image 1601 of FIG. 16. The shaft 110 and/or a tip 114 of the shaft 110 may be delivered into contact with an underside (e.g., proximal side) of the valve leaflet 54. The valve may be, for example, the mitral valve and the leaflet 54 may be, for example, the posterior leaflet of the mitral valve. The proximal side of the valve may be on the ventricle 33 side, as opposed to the atrium 32 (or other chamber) side. The tip 114 of the shaft 110 may have a curved shape and/or may be composed at least partially of an elastic material such that the tip 114 may be configured to at least partially conform to a shape of the leaflet 54.

The shaft 110 may be a single-lumen shaft (see, e.g., FIG. 13) and/or may be configured to deliver multiple needles near-simultaneously and/or prior to removal of the shaft 110 from the ventricle 33. In some instances, the shaft 110 may be positioned at least partially at a region of the leaflet 54 at which there may be relatively little concern of a needle slipping out of the leaflet 54 after puncturing through the leaflet 54. For example, the shaft 110 may be positioned such that an opening of the interior lumen 111 of the shaft 110 is below a point of the leaflet 54 that has a relatively low risk of dislodging a needle.

At block 1504, the process 1500 involves deploying a first needle 130a through the leaflet 54 by extending the first needle 130a out of the shaft 110 and through the leaflet 54, as shown in image 1603 of FIG. 16. In some instances, the first needle 130a may be extended through a reduced width/diameter portion 121 of a lumen 111 of the shaft 110. The reduced width/diameter portion 121 of the lumen 111 may be configured to allow only one needle 130 to pass through the reduced width/diameter portion 121 at a time. The first needle 130a may be advanced and/or extended out of the shaft 110 through use of a plunger configured to advance all three needles 130 and/or through use of a plunger only configured to advance the first needle 130a.

In some instances, the shaft 110 may be positioned such that the first needle 130a pierces the leaflet 54 at a point of the leaflet 54 that is generally flat and/or perpendicular to the shaft 110. The first needle 130a may be extended such that a first sutureform 191a wrapped around the first needle 130a passes entirely through the leaflet 54 before other needles 130 penetrate the leaflet. However, the sutureform 191 may pass only partially through and/or may not enter the leaflet before the shaft 110 is moved and/or other needles 130 penetrate the leaflet 54.

A first pusher 137 may be configured to be extended along the first needle 130a to facilitate removal of the first leaflet anchor 191a from the first needle 130a. In some instances, the first pusher 137a may have a generally hollow interior and/or may comprise an interior lumen to allow the first pusher 137a to slidably receive the first needle 130a and/or to extend along an exterior surface of the first needle 130a. The diameter of the interior lumen of the first pusher 137a may be approximately equivalent to an outer diameter of the first needle 130 such that the first pusher 137a may be configured to press against the first leaflet anchor 191a when the first pusher 137a extends along the first needle 130a. Each of the first needle 130a, first leaflet anchor 191a, first pusher 137a, and/or any number of additional needles 130, leaflet anchors 191, and/or pushers 137 may be configured to be disposed at least partially within an interior lumen of the shaft 110.

In some instances, when the first leaflet anchor 191a is positioned at a distal side of the leaflet 54, the first pusher 137a may be extended to press and/or push the first leaflet anchor 191a off of the first needle 130a. When the first leaflet anchor 191a is removed from the needle 130a, the first leaflet anchor 191a may be configured to naturally form into a knot-type anchor (e.g., a bulky knot), as shown in image 1605 of FIG. 16.

At block 1506, the process involves moving the shaft 110 to situate an opening of the interior lumen 111 and/or a second needle 130b and/or a third needle 130c below a different position and/or different positions of the leaflet 54, as shown in image 1605 of FIG. 16. For example, the shaft 110 may be moved such that the opening into the interior lumen 111, the second needle 130b, and/or the third needle 130c are situated under portions of the leaflet 54 that are nearer the leaflet edge than the penetration point of the first needle 130a. In some instances, a handle of a delivery device may be moved and/or twisted to cause movement of the shaft 110 and/or the shaft 110 may be moved independently of the handle.

The first needle 130a may be retracted back into the shaft 110 before the shaft 110 is moved. The first sutureform 191 and/or first suture anchor may remain anchored to a distal side of the leaflet 54. One or more suture tails 195a of the first sutureform 191a may extend from the first sutureform 191a and/or into the shaft 110. As shown in image 1605 of FIG. 16, the first sutureform 191a may be configured to naturally form into a bulky knot and/or similar suture anchor to prevent the first sutureform 191a from receding back through the leaflet 54 with the first needle 130a.

As the shaft 110 is moved, the first sutureform 191a may hold its position at the distal side of the leaflet 54 while the positions of the second needle 130b and/or third needle 130c may be changed. The shaft 110 may be moved until the first needle 130a is situated further from the leaflet edge than the second needle 130b and/or third needle 130c.

At block 1508, the process involves deploying the second needle 130b and/or second leaflet anchor 191b at least partially through the leaflet 54, as shown in image 1607 of FIG. 16. In some instances, the second needle 130b may be configured to penetrate the leaflet 54 at a point that is nearer the leaflet edge than the penetration point of the first needle 130a. The second needle 130b may be configured to be advanced through the leaflet 54 until the second sutureform 191b is entirely situated at a distal side of the leaflet 54.

In some instances, a second pusher 137b, having an interior lumen to allow the second pusher 137b to extend along the second needle 130b may be configured to extend along the second needle 130b to push the second leaflet anchor 191b off of the second needle 130b when the second leaflet anchor 191b is situated at a distal side of the leaflet 54.

The steps of the process 1500 may be repeated for any number of needles 130. For example, the second needle 130b may be retracted to cause the second leaflet anchor 191b to form into a bulky knot and/or other type of suture anchor. The shaft 110 may be moved again to a different position and/or a third needle 130c may be extended out of the shaft 110 and/or through the leaflet 54 at a different portion of the leaflet 54. Each of the needles 130 may be advanced until the sutureforms 191 on each needle 130 are entirely on the distal side of the leaflet 54 (e.g., the atrial side). A third pusher may be extended along the third needle 130c to push the third leaflet anchor 191c off of the third needle 130c when the third leaflet anchor 191c is situated at a distal side of the leaflet.

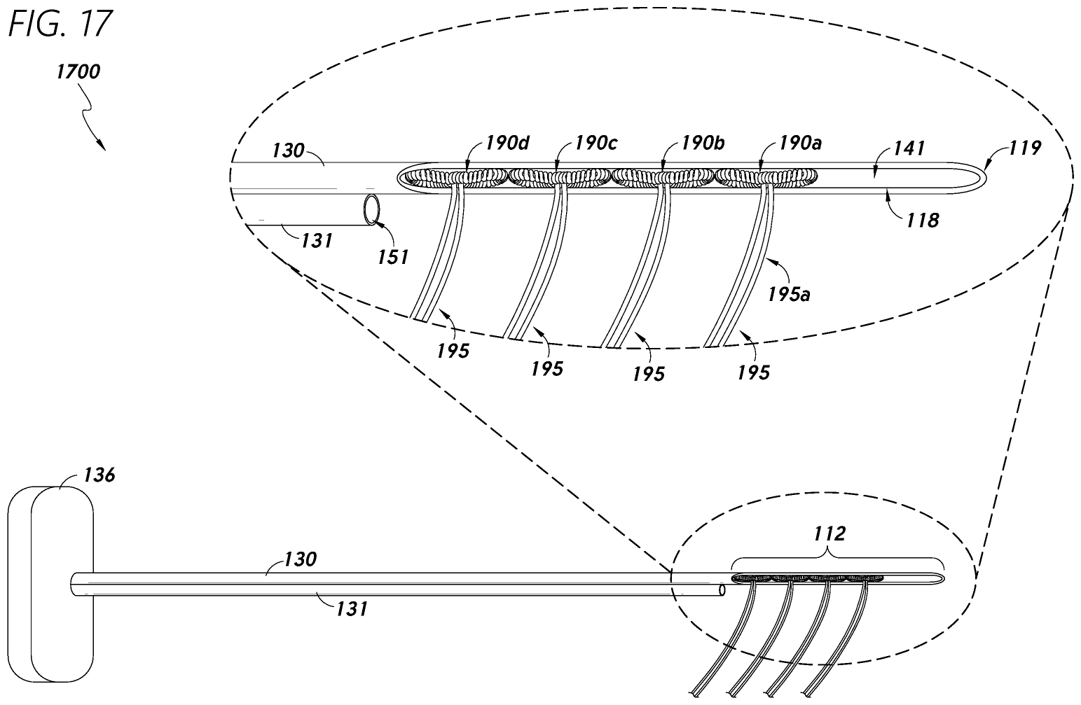

FIG. 17 illustrates an example delivery system 1700 for one or more suture anchors 190 in accordance with one or more instances of the present disclosure. In some instances, one or more sutureforms may be pre-deployed and/or pre-formed into suture anchors 190 (e.g., bulky knot anchors) prior to delivery into a patient's body and/or through a leaflet of a heart valve. One or more suture anchors 190 may be situated within and/or carried via a lumen 141 and/or open portion 112 of a needle 130 for delivery into a heart. For example, at the open portion 112, at least a portion of the needle 130 may be open such that the lumen 141 of the needle 130 may be at least partially exposed. In some instances, the needle 130 may have a semi-circular and/or C-shaped form at the open portion 112. The needle 130 may comprise an opening into the interior lumen 141 at or near a distal tip 119 of the needle 130. The opening may be configured to allow one or more suture anchors 190 to exit the needle 130.

The open portion 112 of the needle 130 may comprise a portion of the needle 130 having an elongate aperture 118 and/or slot extending longitudinally along a first side of the needle 130. The elongate aperture 118 may be configured and/or sized to allow one or more suture tails 195 extending from the one or more suture anchors 190 to exit the lumen 141 of the needle 130. However, the elongate aperture 118 may not be sufficiently wide to allow the one or more suture anchors 190 to entirely exit the needle until the one or more suture anchors 190 extend to the opening at the distal end 119 of the needle 130. In other words, the elongate aperture 118 may be configured to prevent the one or more suture anchors 190 from exiting the needle via the elongate aperture 118 while allowing individual suture tails 195 to exit the needle 130 via the elongate aperture 118. For example, the elongate aperture 118 may have a width that is less than a maximal width of the one or more suture anchors 190 but greater than a width of a single suture tail 195.

One or more suture anchors 190 may be situated within the needle 130 (e.g., at the open portion 112 of the needle 130). In some instances, the suture anchors 190 may be sized, shaped, and/or positioned such that the one or more suture anchors 190 may not become dislodged from the needle 130 until the suture anchors 190 are deployed at a distal tip 119 of the needle 130. For example, the one or more suture anchors 190 may be configured to slide along the needle 130 and/or the open portion 112 of the needle 130 in response to pushing force (e.g., from a pusher and/or similar device) without exiting the needle 130 through the open side of the needle 130. The suture anchors 190 may extend into suture tails 195 which may be configured to extend out of the open portion 112 of the needle 130. For example, one or more suture tails 195 may be accessible to a physician.

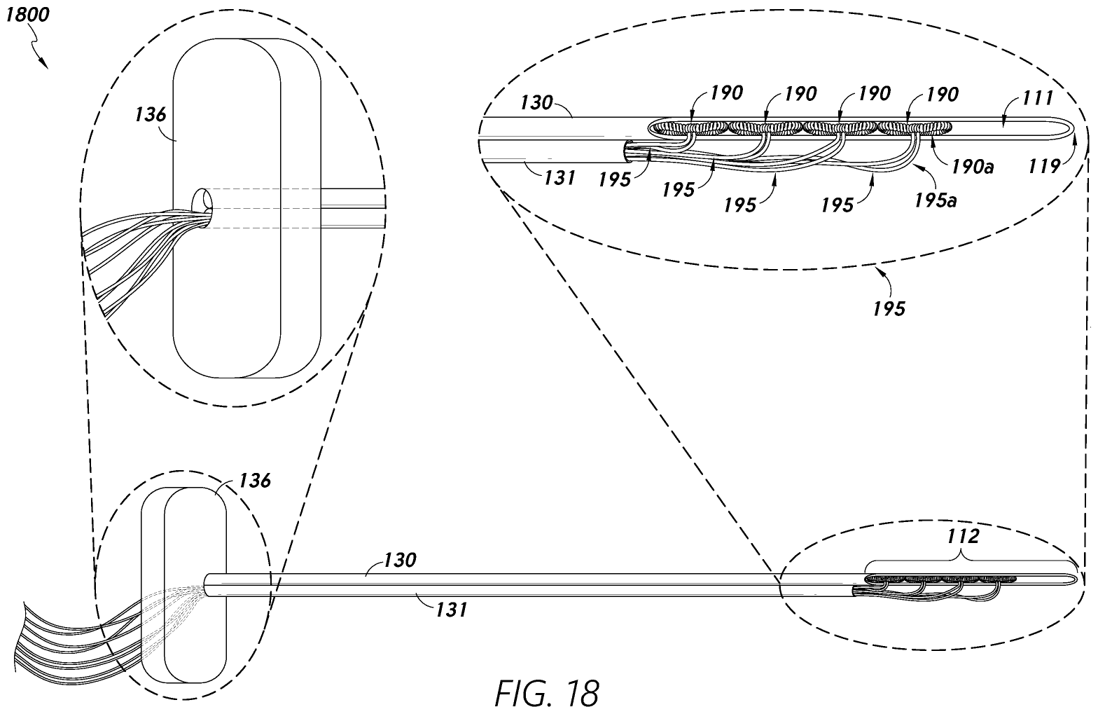

In some instances, the delivery system 1700 may comprise a support shaft 131 (e.g., a needle) configured to extend in parallel with at least a portion of the needle 130. The support shaft 131 may be configured to receive one or more suture tails 195 extending from the suture anchors 190 (see, e.g., FIG. 18) into an interior lumen 151 of the support shaft 131. In some instances, the needle 130 and/or support shaft 131 may be configured to extend to and/or through a handle 136 and/or similar device. While a rectangular handle 136 is shown in FIGS. 17 and 18, the handle 136 may include and/or be replaced by the various delivery devices described herein (e.g., the delivery device 330 shown in FIGS. 14A and/or 14B).

The one or more suture anchors 190 may be configured to be oriented and/or situated in a generally single-file line within the lumen 141 of the needle 130. For example, the lumen 141 may have a diameter approximately equal to diameters of the suture anchors 190 such that the suture anchors 190 are oriented in a line within the lumen 141. Accordingly, the one or more suture anchors 190 may be configured to press against each other when force is applied to one of the suture anchors 190. For example, a pusher may be extended within the needle 130 to press against a fourth suture anchor 190d (e.g., a proximal suture anchor). Force from the pusher against the fourth suture anchor 190d may cause the fourth suture anchor 190d to press against a third suture anchor 190c, which may cause the third suture anchor 190c to press against a second suture anchor 190b, which may cause the second suture anchor 190b to press against a first suture anchor 190a to move the one or more suture anchors 190 toward the distal end 119 of the needle 130 and/or an opening at the distal end 119 of the needle 130.

FIG. 18 illustrates another example delivery system 1800 for one or more suture anchors 190 in accordance with one or more instances of the present disclosure. In some instances, one or more suture tails 195 may be configured to exit an open portion 112 of a needle 130 and/or enter a support shaft 131. The support shaft 131 may be situated in parallel and/or at least partially in contact with the needle 130. In this way, the one or more suture tails 195 may be configured to pass through the support shaft 131 and/or may be accessible to a physician.

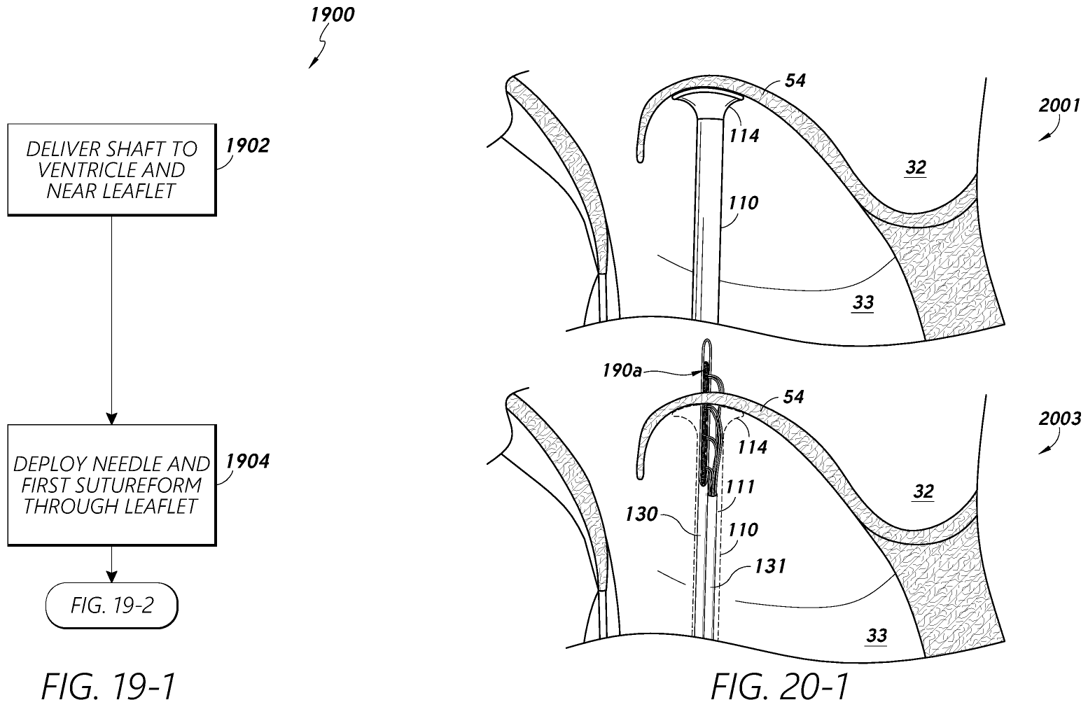

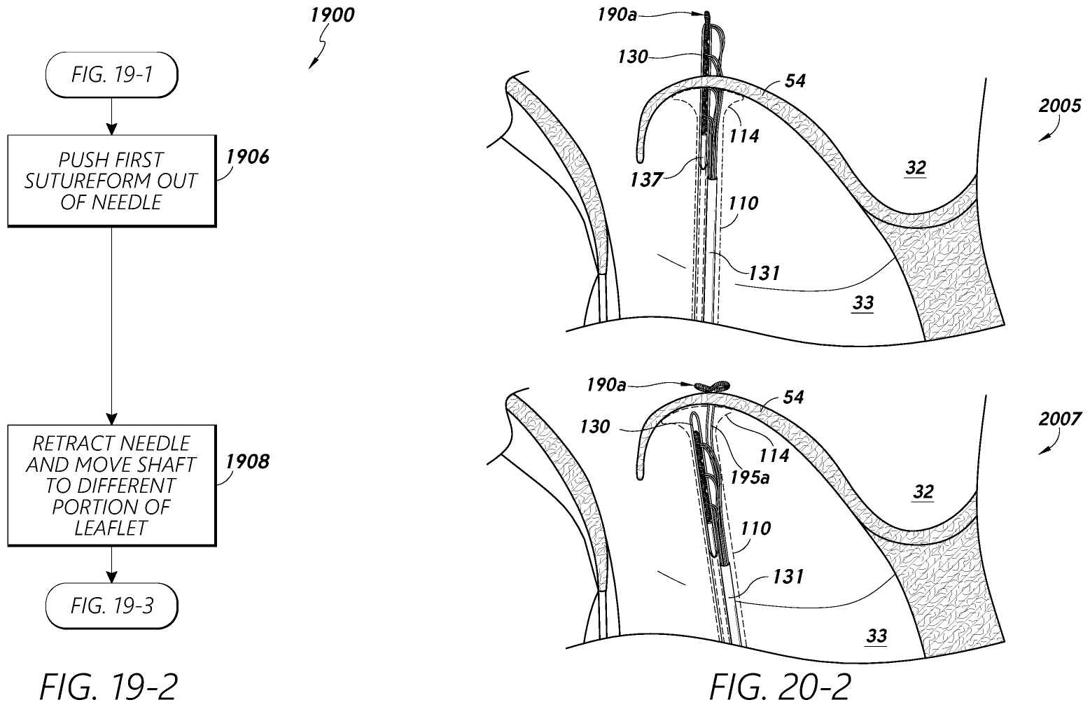

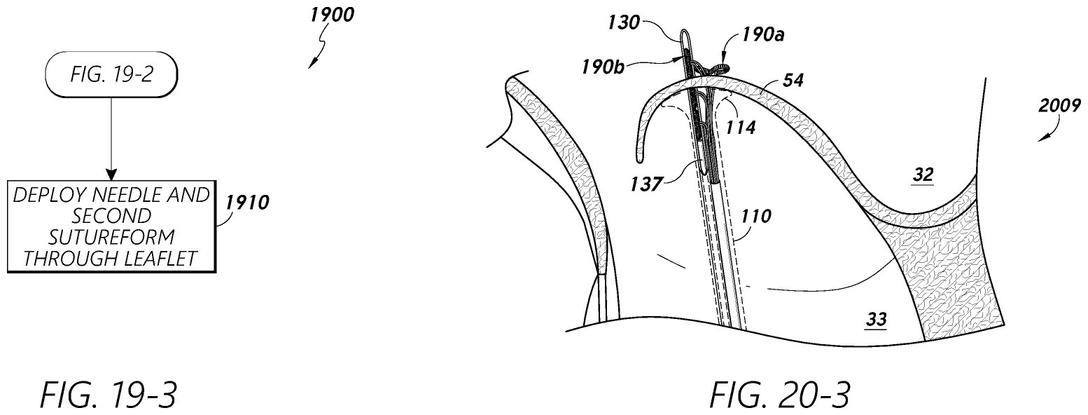

FIG. 19 (FIGS. 19-1 and 19-2) is a flow diagram illustrating a process 1900 for delivering multiple leaflet anchors to a valve leaflet in accordance with one or more instances of the present disclosure. FIG. 20 (FIGS. 20-1 and 20-2) provides illustrations corresponding to steps of the process 1900 described in FIG. 19.

At step 1902, the process 1900 involves delivering a shaft 110 to a ventricle 33 (e.g., the left ventricle) and/or at or near a leaflet 54 of a valve of the ventricle 33 (e.g., the mitral valve), as shown in image 2001 of FIG. 20. In some instances, the shaft 110 may be sized and/or shaped to deliver one or more needles, support shafts, and/or anchors (e.g., suture anchors) to the leaflet 54. The shaft 110 may comprise a tip portion 114 (e.g., an atraumatic tip) configured to contact the leaflet 54 without damaging the leaflet 54.

At step 1904, the process 1900 involves deploying a needle 130 and/or first suture anchor 190a (e.g., suture anchor) situated within a lumen (e.g., at an open portion) of the needle 130 through the leaflet 54 and/or to a distal side of the leaflet 54, as shown in image 2003 of FIG. 20. The needle 130 and/or a support shaft 131 may be situated within the shaft 110 and/or may be configured to slide along an inner lumen of the shaft 110. The needle 130 may be configured to penetrate and/or pass through any suitable portion of the leaflet 54.

At step 1906, the process 1900 involves pushing and/or extending the first suture anchor 190a along the needle 130 until the first suture anchor 190a exits the needle 130 (e.g., at a distal tip portion of the needle 130), as shown in image 2005 of FIG. 20. In some instances, a pusher 137 and/or similar device may be configured to apply pushing force to the first suture anchor 190a and/or other sutureforms within the needle 130. For example, multiple sutureforms may be situated within the needle 130 and/or the pusher 137 may be configured to press against a proximal sutureform to thereby apply pushing force to each of the sutureforms, including the first suture anchor 190a. After the first suture anchor 190a exits the needle 130 and/or before any additional sutureforms exit the needle 130, the pusher 137 may be retracted and/or may stop applying pushing force to the sutureforms.

At step 1908, the process 1900 involves retracting the needle 130 and/or moving the needle 130 and/or shaft 110 to position the needle 130 and/or shaft 110 below and/or at a different portion of the leaflet 54, as shown in image 2007 of FIG. 20. For example, the needle 130 may be retracted such that the needle 130 completely exits the leaflet 54 prior to moving the needle 130 to a different portion of the leaflet 54. However, the needle 130 and/or shaft 110 may not need to be removed from the body, as the additional sutureforms may be configured for delivery immediately following delivery of the first suture anchor 190a. In some instances, the tip portion 114 may remain in contact with the leaflet 54 as the needle 130 and/or shaft 110 are moved.

The first suture anchor 190a may comprise any of a variety of forms including a “bulky knot” form which may prevent the first suture anchor 190a from passing through the leaflet 54. Thus, the first suture anchor 190a may remain at a distal side of the leaflet 54 while the needle 130 is retracted and/or moved. One or more suture tails 195a of the first suture anchor 190a may be configured to pass through an opening in the leaflet 54 created by the needle 130 and/or may extend into the support shaft 131 and/or the into the shaft 110.

At step 1910, the process 1900 involves deploying the needle 130 and/or a second suture anchor 190b through the leaflet 54 at the different portion of the leaflet 54, as shown in image 2009 of FIG. 20. The needle 130 may be configured to puncture the leaflet 54 at each portion of the leaflet 54 to create an opening through which the needle and/or suture tails 195 can pass. In some instances, the needle 130 may puncture the leaflet 54 at a second portion that is a suitable distance from the anchor point of the first suture anchor 190a. For example, the second suture anchor 190b may be deployed at point of the leaflet 54 such that the second suture anchor 190b does not contact the first suture anchor 190a following delivery of the second suture anchor 190b at the distal side of the leaflet 54.

The steps of the process 1900 may be repeated as necessary for delivery of any number of suture anchors 190. For example, the second suture anchor 190b may be pressed out of the needle 130 using a pusher 137 and/or similar device. The needle may be retracted from the leaflet 54 and/or moved to a third portion of the leaflet 54 for delivery of a third sutureform. Any number of sutureforms may be delivered at a distal side of the leaflet in this manner.

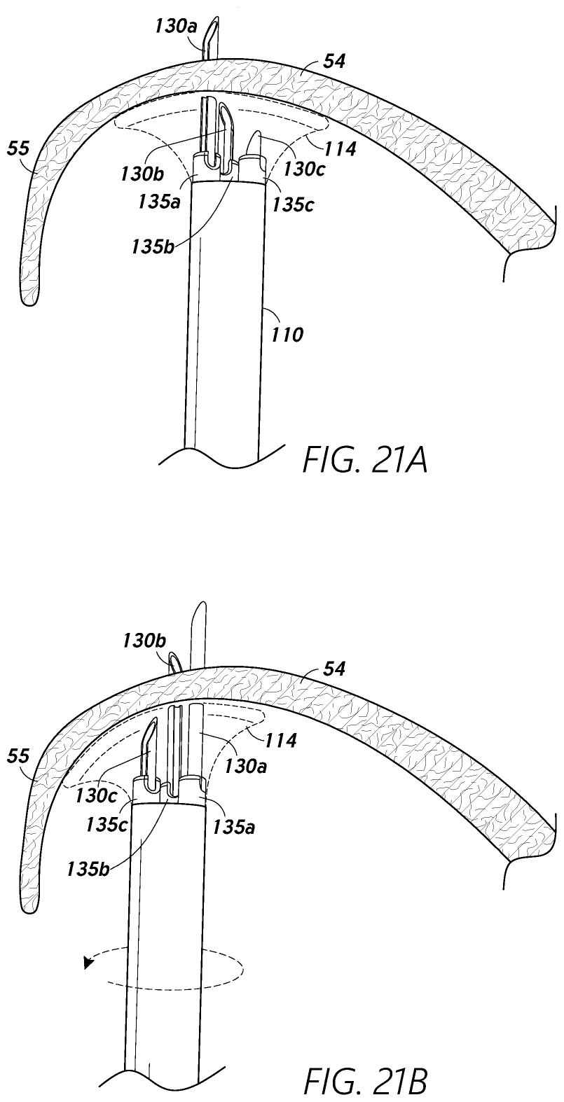

FIG. 21A illustrates a multi-lumen shaft 110 delivering multiple needles to a valve leaflet 54. A leaflet generally comprises an edge portion 55 (or “free edge”) at an end portion of the leaflet 54. In healthy valve, the edge portion 55 may be the main area of chordal attachment and/or the region of coaptation with another valve leaflet. For example, an edge portion of a posterior valve leaflet may coapt with a corresponding edge portion of an anterior valve leaflet. Thus, it may be advantageous to deliver one or more suture anchors at or near the edge portion 55 of the leaflet 54 to allow the suture ends of the suture anchor to most closely approximate performance of the chordae tendineae.

However, the shape and/or movement of the edge portion 55 may present difficulties in anchoring to the edge portion 55. For example, after a needle penetrates the edge portion 55 and/or a suture anchor is delivered to a distal side of the edge portion 55, the needle and/or suture anchor may be susceptible to slipping out of contact with the leaflet, particular during beating heart procedures.

The shaft 110 may be configured to carry one or more needles 130 (e.g., a first needle 130a, a second needle 130b, and/or a third needle 130c). In some embodiments, the needles 130 may be spaced around an inner diameter of the shaft 110. One or more of the needles 130 may delivered via sheaths 135 configured to at least partially enclose the needles 130. For example, a first sheath 135a may be configured to enclose the first needle 130a, a second sheath 135b may be configured to enclose the second needle 130b, and/or a third sheath 135c may be configured to enclose the third needle 130c.

FIG. 21B illustrates rotation of the multi-lumen shaft 110 to facilitate anchoring at or near the leaflet edge 55. In some examples, the first needle 130a delivered via the shaft 110 may be extended to pierce the leaflet 54 at a first point. The first needle 130a may be longer than the second needle 130b and/or third needle 130c and/or the first needle 130a may be extended further than the second needle and/or third needle 130c such that the first needle 130a pierces the leaflet 54 before the second needle 130b and third needle 130c pierce the leaflet 54.

After the first needle 130a pierces the leaflet, the shaft 110 may be twisted and/or rotated until the second needle 130b and/or third needle 130c is below a desired position of the leaflet 54 that is nearer the leaflet edge 55 than the first point. In some examples, the second needle 130b and/or third needle 130c may be configured to pierce the leaflet at approximately ½ mm to 3 mm from the leaflet edge 55. The first point may be a greater distance from the leaflet edge 55 than 0.5 mm to 3 mm, and may be approximately 5 mm or 6 mm or more from the leaflet edge 55.

By piercing the leaflet 54 first with the first needle 130a, the first needle 130a may be configured to maintain contact between the delivery systems and the leaflet 54 to more easily anchor other needles at or near the leaflet edge 55. Because the first needle 130a can penetrate the leaflet 54 at a greater distance from the leaflet edge 55 than the second needle 130b and/or third needle 130c, the first needle 130a may be less likely to slip out of contact with the leaflet 54 after delivery of the first needle 130a through the leaflet 54.

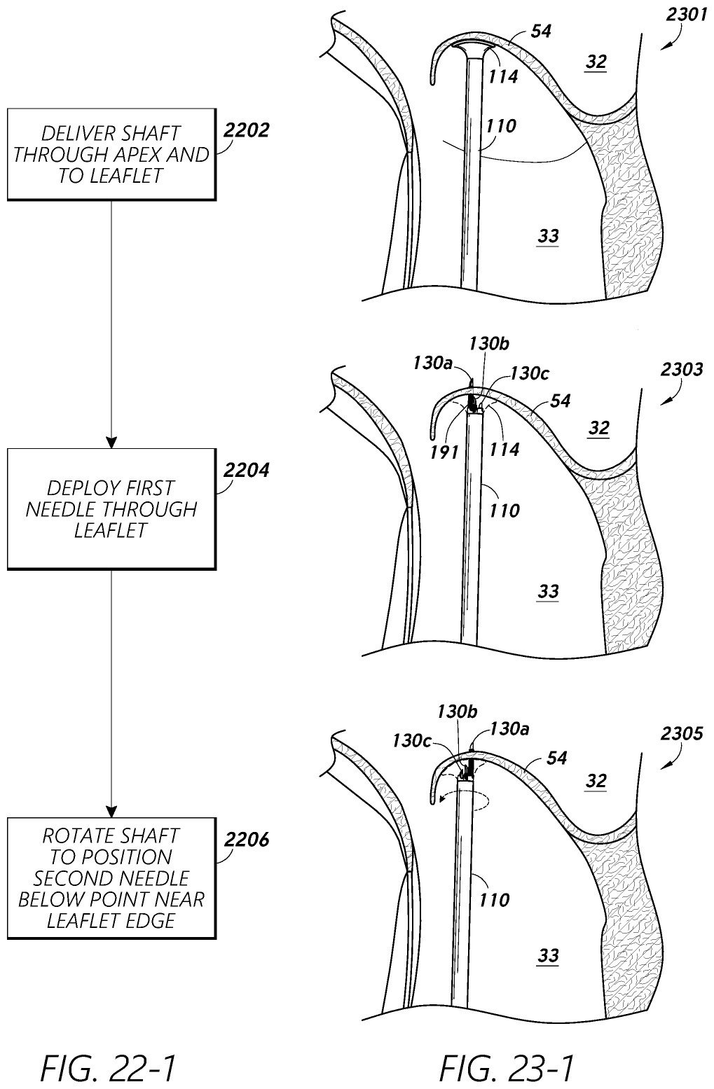

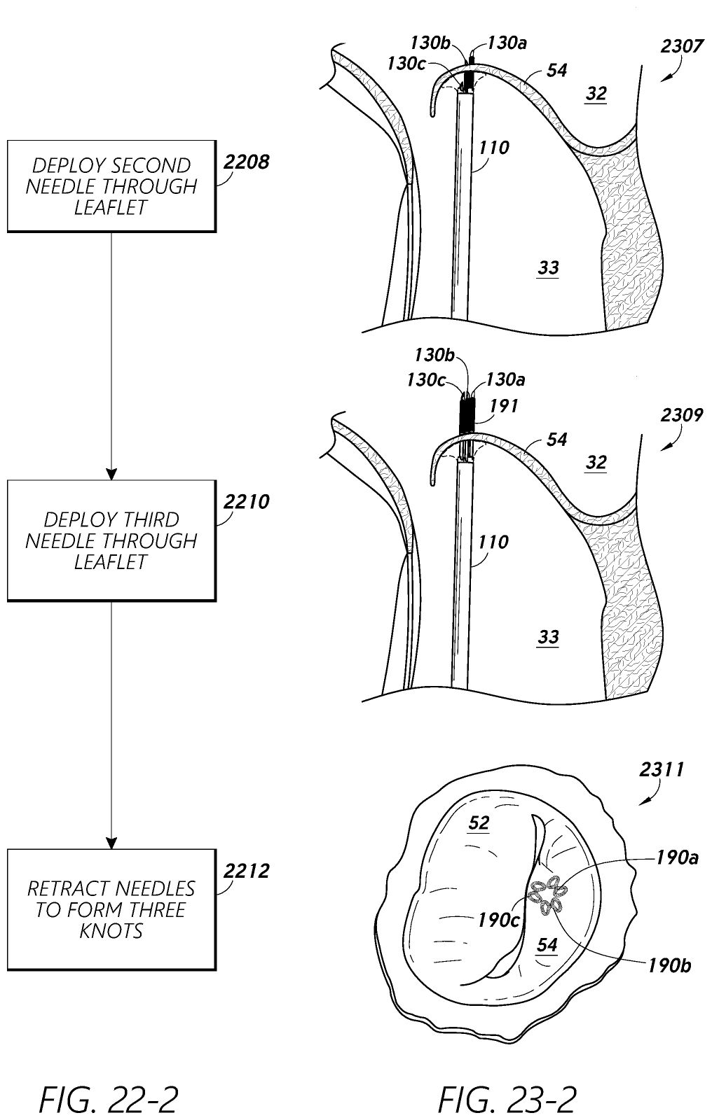

FIG. 22 (e.g., FIGS. 22-1 and 22-2) is a flow diagram illustrating a process for delivering multiple leaflet anchors to a valve leaflet in accordance with one or more instances of the present disclosure. FIG. 23 (e.g., FIGS. 23-1 and 23-2) provides illustrations corresponding to steps of the process described in FIG. 22.

At block 2202, the process involves delivering a shaft of a delivery device (see FIG. 2) through a wall of a heart (e.g., the apex region) into a ventricle 33 and into contact with a leaflet 54 of a valve of the heart. As shown in image 2301 of FIG. 23, the shaft 110 and/or a tip 114 of the shaft 110 may be delivered into contact with an underside (e.g., proximal side) of the valve leaflet 54. The valve may be, for example, the mitral valve and the leaflet 54 may be, for example, the posterior leaflet of the mitral valve. The proximal side of the valve may be on the ventricle 33 side, as opposed to the atrial 32 (or other chamber) side. The tip 114 of the shaft 110 may have a curved shape and/or may be composed at least partially of an elastic material such that the tip 114 may be configured to at least partially conform to a shape of the leaflet 54.

The shaft 110 may be a multi-lumen shaft (see FIG. 10A) and/or may be configured to deliver multiple needles simultaneously and/or prior to being removed from the ventricle 33. In some instances, the shaft 110 may be positioned at least partially at a region of the leaflet 54 at which there may be relatively little concern of a needle slipping out of the leaflet 54 after puncturing through the leaflet 54. For example, the shaft 110 may be positioned such that at least a first lumen of the shaft 110 is below a point of the leaflet 54 that has a relatively low risk of dislodging a needle.

At block 2204, the process involves deploying a first needle 130a through the leaflet 54 (see image 2303 of FIG. 23) by extending the first needle 130a out of the shaft 110 and through the leaflet 54. In some instances, the first needle 130a may be longer and/or positioned further in the shaft 110 than a second needle 130b and/or third needle 130c situated within other lumens of the shaft 110. The first needle 130a may be advanced and/or extended out of the shaft 110 through use of a plunger configured to advance all three needles 130 and/or through use of a plunger only configured to advance the first needle 130a.

In some embodiments, the shaft 110 may be positioned such that the first needle 130a pierces the leaflet 54 at a point of the leaflet 54 that is generally flat and/or perpendicular to the shaft 110. The first needle 130a may be extended such that a sutureform 191 wrapped around the first needle 130a passes entirely through the leaflet 54 before other needles 130 penetrate the leaflet. However, the sutureform 191 may pass only partially through and/or may not enter the leaflet before the shaft 110 is rotated and/or other needles 130 penetrate the leaflet 54.

At block 2206, the process involves rotating the shaft 110 to situate a second needle 130b and/or a third needle 130c below a position and/or positions of the leaflet 54 that are nearer the leaflet edge than the penetration point of the first needle 130a (see image 2305 of FIG. 23). In some instances, a handle of a delivery device may be twisted to cause twisting of the shaft 110 and/or the shaft 110 may be twisted independently of the handle.

As the shaft 110 is twisted, the first needle 130a may hold its position within the leaflet 54 while the positions of the second needle 130b and/or third needle 130c may be changed. The shaft 110 may be twisted until the first needle 130a is situated further from the leaflet edge than the second needle 130b and/or third needle 130c. The second needle 130b and/or third needle 130c may be extended out of the shaft 110, but not in contact with the leaflet 54, when the shaft 110 is rotated.

At block 2208, the process involves deploying the second needle 130b at least partially through the leaflet 54 (see image 2307 of FIG. 23). The second needle 130b may penetrate the leaflet 54 at a point that is nearer the leaflet edge than the penetration point of the first needle 130a.

At block 2210, the process involves deploying the third needle 130c at least partially through the leaflet 54 (see image 2309 of FIG. 23). The third needle 130c may penetrate the leaflet 54 at a point that is nearer the leaflet edge than the penetration point of the first needle 130a and/or second needle 130b and/or may penetrate the leaflet 54 at a point that is further from the leaflet edge than the first needle 130a and/or second needle 130b. Each of the needles 130 may be advanced until the sutureforms 191 on each needle 130 are entirely on the distal side of the leaflet 54 (e.g., the atrial 32 side).

At block 2212, the process involves retracting the needles 130 to form the bulky knot anchors 190 (see image 2311 of FIG. 23). The needles 130 may be retracted through pushers while the pushers remain in place to prevent the sutureforms 191 from passing back through the leaflet 54 and into the ventricle 33. The needles 130 may be configured to press the sutureforms 191 against the pushers to form the bulky knot anchors 190. A first bulky knot anchor delivered via the first needle 130a may be further from the leaflet edge than a second bulky knot anchor 190b and/or a third bulky knot anchor 190c.

### Additional Examples

Depending on the example, certain acts, events, or functions of any of the processes or algorithms described herein can be performed in a different sequence, may be added, merged, or left out altogether. Thus, in certain instances, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain instances include, while other instances do not include, certain features, elements and/or steps. Thus, such conditional language is not generally intended to imply that features, elements and/or steps are in anyway required for one or more instances or that one or more instances necessarily include logic for deciding, with or without author input or prompting, whether these features, elements and/or steps are included or are to be performed in any particular instance. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain instances require at least one of X, at least one of Y and at least one of Z to each be present.

It should be appreciated that in the above description of instances, various features are sometimes grouped together in a single instance, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular instance herein can be applied to or used with any other instance(s). Further, no component, feature, step, or group of components, features, or steps are necessary or indispensable for each instance. Thus, it is intended that the scope of the inventions herein disclosed and claimed below should not be limited by the particular instances described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example instances belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A delivery system comprising:
two or more needles including at least a first needle comprising a distal tip configured to pierce a valve leaflet of a heart;
two or more leaflet anchors, each of the two or more leaflet anchors configured to be delivered via one of the two or more needles; and
a shaft comprising a first interior lumen configured to slidably receive the two or more needles, the shaft having a first opening into the first interior lumen at a distal end of the shaft, the first opening configured to allow at least the first needle to exit the shaft.

2. The delivery system of claim 1, wherein the first interior lumen has a non-constant diameter that gradually decreases from a maximal diameter of the first interior lumen to a minimal diameter at the first opening that is less than the maximal diameter of the first interior lumen.

3. The delivery system of claim 2, wherein the first opening is configured to allow only one of the two or more needles to exit the shaft at a time.

4. The delivery system of claim 1, wherein a first leaflet anchor of the two or more leaflet anchors is configured to be delivered via the first needle and a second leaflet anchor of the two or more leaflet anchors is configured to be delivered via a second needle of the two or more needles.

5. The delivery system of claim 4, wherein each of the two or more leaflet anchors comprises a suture wrapped at least partially around a distal portion of one of the two or more needles and configured to form a knot anchor when removed from the two or more needles.

6. The delivery system of claim 5, further comprising two or more pushers, each of the two or more pushers having interior lumens and configured to:
be disposed at least partially within the first interior lumen of the shaft;
slidably receive one of the two or more needles; and
press against one of the two or more leaflet anchors to remove the leaflet anchor from one of the two or more needles.

7. The delivery system of claim 1, further comprising a plunger configured to move the two or more needles out of the shaft.

8. The delivery system of claim 1, wherein the first needle comprises a second interior lumen and a second opening into the second interior lumen at the distal tip of the first needle, the second interior lumen configured to carry the two or more leaflet anchors.

9. The delivery system of claim 8, wherein the first needle comprises an elongate aperture into the second interior lumen, the elongate aperture extending along at least a portion of a first side of the first needle to the second opening and configured to allow one or more suture tails of the two or more leaflet anchors to exit the first needle via the elongate aperture.

10. The delivery system of claim 9, wherein the elongate aperture is configured to prevent the two or more leaflet anchors from entirely exiting the first needle via the elongate aperture.

11. The delivery system of claim 9, wherein a second needle of the two or more needles comprises a third interior lumen configured to receive the one or more suture tails.

12. The delivery system of claim 8, wherein the two or more leaflet anchors are pre-formed suture knots.

13. The delivery system of claim 8, wherein the two or more leaflet anchors are configured to be situated in a single-file orientation within the first needle.

14. A method for simulating treatment of a simulated patient, the method comprising:
delivering a shaft carrying two or more needles to a first portion of a leaflet of a heart ventricle, the shaft comprising a first interior lumen having a non-constant diameter that gradually decreases near an opening into the first interior lumen at a distal end of the shaft to prevent multiple of the two or more needles from exiting the shaft simultaneously at the opening;
deploying a first needle of the two or more needles through the leaflet, the first needle carrying a first sutureform;
extending a first pusher along the first needle to remove the first sutureform from the first needle and to cause the first sutureform to form a first suture knot;
retracting the first needle into the first interior lumen;
moving the shaft to a second portion of the leaflet;
deploying a second needle of the two or more needles through the leaflet, the second needle carrying a second sutureform;
extending a second pusher along the second needle to remove the second sutureform from the second needle and to cause the second sutureform to form a second suture knot; and
retracting the second needle into the first interior lumen.

15. The method of claim 14, wherein the first needle is configured to carry the first sutureform at an exterior surface of the first needle.

16. The method of claim 14, wherein the first needle comprises a second interior lumen and is configured to carry the first sutureform within the second interior lumen.

17. The method of claim 16, wherein the first needle comprises a longitudinal slot into the second interior lumen along at least a portion of a first side of the first needle.

18. A method for simulating treatment of a simulated patient, the method comprising:
delivering a shaft carrying two or more needles to a first portion of a leaflet of a heart ventricle, a first needle of the two or more needles carrying two or more suture anchors within a first interior lumen of the first needle, the first needle comprising an elongate aperture extending along at least a portion of a first side of the first needle to an opening at a distal tip of the first needle, the elongate aperture configured to allow one or more suture tails of the two or more suture anchors to exit the first interior lumen, and a second needle of the two or more needles configured to receive the one or more suture tails into a second interior lumen of the second needle;
deploying the first needle through the leaflet;
extending a pusher along the first needle to press against at least one of the two or more suture anchors to remove a first suture anchor of the two or more suture anchors from the first needle;
retracting the first needle from the leaflet;
moving the shaft to a second portion of the leaflet;
deploying the first needle through the leaflet;
extending the pusher along the first needle to press against at least one of the two or more suture anchors to remove a second suture anchor of the two or more suture anchors from the first needle; and
retracting the first needle out of the leaflet.

19. The method of claim 18, the shaft comprising a third interior lumen having a non-constant diameter that gradually decreases near a second opening into the third interior lumen at a distal end of the shaft to prevent multiple of the two or more needles from exiting the shaft simultaneously at the second opening.

20. The method of claim 18, further comprising deploying a third needle of the two or more needles through the leaflet, the third needle configured to carry two or more suture anchors.
